(19) United States
(12) Patent Application Publication
(10) Pub. No.: US 2018/0345046 A1
(43) Pub. Date: Dec. 6, 2018

(54) CATHETER AND METHOD FOR USE

(71) Applicant: David A. GALLUP, Brentwood, CA (US)

(72) Inventor: David A. GALLUP, Brentwood, CA (US)

(21) Appl. No.: 15/608,583

(22) Filed: May 30, 2017

(51) Int. Cl. A61N 7/02

(52) U.S. Cl. CPC A61N 7/02; A61N 2007/003

(57) ABSTRACT

Tissue ablation systems and methods of using the same are disclosed. The tissue ablation systems can have an ultrasound ablation device connected to a catheter. The ultrasound ablation device can have a first conical reflector having a first reflective surface and a second conical reflector having a second reflective surface. The distance between the first and second conical reflectors can be adjusted, e.g., increased and/or decreased, to adjust the radial and/or longitudinal dimension of a focal zone from the device.

### BACKGROUND

The present disclosure relates generally to tissue ablation and more particularly to ultrasound energy tissue ablation.

Hypertension and other conditions can be treated by ablating nerves, for example, renal nerves. Current tissue ablation devices, including renal denervation devices, use RF, cryogenic, or ultrasound energy sources. However, these current devices have a limited ability to adjust the focal point of the energy delivered, if at all, and as a result there is a high risk of vessel stenosis following the application of therapy.

Relative to current devices, the devices and methods disclosed herein can better control the energy delivered to targeted tissue because it can focus ultrasound energy at different tissue depths. The devices and methods disclosed herein can advantageously deliver a toroid of energy having an adjustable radius from the center of the device. This adjustability can prevent or otherwise minimize vessel stenosis since the device can focus the energy beyond the vessel wall, thereby reducing the risk of damaging non targeted tissue. The devices and methods disclosed herein can advantageously provide safer and faster tissue ablation procedures with improved efficacy.

### BRIEF SUMMARY OF THE INVENTION

Tissue ablation systems and methods of using the same are disclosed.

The tissue ablation systems can have an ultrasound ablation device connected to a catheter.

The ultrasound ablation device can have a longitudinal axis. The ultrasound ablation device can have a first emitter surface and a first conical reflector having a first reflective surface. The first conical reflector can be at a first longitudinal end of the device.

The device can have a catheter, a second emitter surface, and a second conical reflector. The second conical reflector can have a second reflective surface. The second conical reflector can be at a second longitudinal end of the device. The device can have a cylindrical shield. At least a portion of the cylindrical shield can be transparent such that less than 10% of ultrasound energy that passes therethrough is attenuated. The device can have an ultrasound coupling chamber. The first and second emitter surfaces, the first and second reflective surfaces, and a coupling fluid can be in the ultrasound coupling chamber. The coupling fluid can be saline. The ultrasound coupling chamber can have a coupling fluid inlet and a coupling fluid first outlet in fluid communication with the ultrasound coupling chamber. The ultrasound coupling chamber can be at least partially defined by the cylindrical shield. The first and second emitter surfaces can be flat. An axis normal to the first emitter surface can extend at least partially in a first longitudinal direction toward the first longitudinal end of the device. An axis normal to the second emitter surface can extend at least partially in a second longitudinal direction toward the second longitudinal end of the device. The first and second emitter surfaces can be surfaces of a piezoelectric transducer configured to have a harmonic frequency from 9 MHz to 15 MHz. The first reflective surface can extend around the longitudinal axis. The second reflective surface can extend around the longitudinal axis. The first reflective surface can be at a first reflector surface angle relative to the longitudinal axis. The first reflector surface angle can be from 30 degrees to 60 degrees. The second reflective surface can be at a second reflector surface angle relative to the longitudinal axis. The second reflector surface angle can be from 30 degrees to 60 degrees. The device can have a first configuration and a second configuration. At least one of the first and second conical reflectors can be closer to at least one of the first and second emitter surfaces in the first configuration than in the second configuration.

The coupling fluid can be saline.

The ultrasound coupling chamber can be at least partially defined by a shield that extends at least partially around the longitudinal axis.

At least a portion of the shield is transparent such that less than 10% of ultrasound energy that passes therethrough is attenuated.

The device can have a coupling fluid inlet and a coupling fluid first outlet in fluid communication with the ultrasound coupling chamber.

A coupling fluid source can be in fluid communication with the coupling fluid inlet.

The coupling fluid first outlet can be a recirculation port in fluid communication with the coupling fluid source and/or another coupling fluid source.

The coupling fluid first outlet can be in fluid communication with an outside environs.

The device can have a coupling fluid second outlet in fluid communication with the ultrasound coupling chamber. The coupling fluid first outlet can be a recirculation port in fluid communication with the coupling fluid source and/or another coupling fluid source. The coupling fluid second outlet can be in fluid communication with an outside environs.

At least one of the first and second conical reflectors can be movable relative to at least one of the first and second emitter surfaces. At least one of the first and second emitter surfaces can be movable relative to at least one of the first and second conical reflectors. The first and second conical reflectors and the first and second emitter surfaces can each be movable relative to one another.

At least one of the first and second conical reflectors can be movable relative to at least one of the first and second emitter surfaces at least partially in a longitudinal direction. At least one of the first and second emitter surfaces can be movable relative to at least one of the first and second conical reflectors at least partially in a longitudinal direction. The first and second conical reflectors and the first and second emitter surfaces can each be movable relative to one another at least partially in a longitudinal direction.

The device can have a first shaft and a second shaft. The first conical reflector can be attached to the first shaft and the second conical reflector can be attached to the second shaft. The first or second shaft can be slideable within or over the second or first shaft, respectively.

The ultrasound ablation device can have a longitudinal axis. The device can have a transducer having a first emitter surface and a first conical reflector having a first reflective surface. The first conical reflector can be at a first longitudinal end of the device.

The first reflective surface can extend at least partially around the longitudinal axis.

The first reflective surface can be at a first reflector surface angle relative to the longitudinal axis. The first reflector surface angle can be from 0 degrees to 90 degrees.

The transducer can be a disc or an annular disc.

The transducer can be a piezoelectric transducer.

The first emitter surface can be flat.

The first emitter surface can be at a first emitter surface angle relative to the longitudinal axis. The first emitter surface angle is from 0 degrees to 90 degrees.

The device can have a first configuration and a second configuration. The first conical reflector can be closer to the transducer along the longitudinal axis in the first configuration than in the second configuration.

The device can have a shaft having a shaft first end and a shaft second end. The transducer can be attached to or integrated with the shaft first end. The first conical reflector can be attached to or integrated with the shaft second end.

At least a portion of the first conical reflector can be movable relative to the transducer. At least a portion of the transducer can be movable relative to the first conical reflector. Both the transducer and the first conical reflector can be movable relative to one another.

The first conical reflector can be movable along the longitudinal axis relative to the transducer. The transducer can be movable along the longitudinal axis relative to the first conical reflector. Both the transducer and the first conical reflector can be movable along the longitudinal axis relative to one another.

The device can be integrated with or attached to a catheter.

The device can have a second emitter surface and a second conical reflector having a second reflective surface. The second conical reflector can be at a second longitudinal end of the device.

The first reflective surface can extend at least partially around the longitudinal axis. The second reflective surface can extend at least partially around the longitudinal axis.

The first reflective surface can be at a first reflector surface angle relative to the longitudinal axis. The first reflector surface angle can be from 0 degrees to 90 degrees. The second reflective surface can be at a second reflector surface angle relative to the longitudinal axis. The second reflector surface angle can be from 0 degrees to 90 degrees.

The first conical reflector can be at a first longitudinal terminal end of the device. The second conical reflector can be at a second longitudinal terminal end of the device.

The transducer can have the second emitter surface.

The first and second emitter surfaces can be perpendicular to the longitudinal axis and face away from each other in opposite directions.

The transducer can be a disc or an annular disc.

The transducer can be a piezoelectric transducer.

At least one of the first emitter surface and the second emitter surface can be flat.

The first emitter surface can be at a first emitter surface angle relative to the longitudinal axis. The first emitter surface angle can be from 0 degrees to 90 degrees. The second emitter surface can be at a second emitter surface angle relative to the longitudinal axis. The second emitter surface angle can be from 0 degrees to 90 degrees.

The device can have a first configuration and a second configuration. At least one of the first conical reflector and the second conical reflector can be closer to the transducer along the longitudinal axis in the first configuration than in the second configuration.

The device can have a shaft having a shaft first end and a shaft second end. The transducer can be attached to or integrated with the shaft. The first conical reflector can be attached to or integrated with the shaft first end. The second conical reflector can be attached to or integrated with the shaft second end.

At least a portion of at least one of the first and second conical reflectors can be movable relative to at least one of the first and second emitter surfaces. At least one of the first and second emitter surfaces can be movable relative to at least one of the first and second conical reflectors. The first and second conical reflectors and the first and second emitter surfaces can each be movable relative to one another.

At least a portion of at least one of the first and second conical reflectors can be movable along the longitudinal axis relative to at least one of the first and second emitter surfaces. At least one of the first and second emitter surfaces can be movable along the longitudinal axis relative to at least one of the first and second conical reflectors. The first and second conical reflectors and the first and second emitter surfaces can each be movable along the longitudinal axis relative to one another.

The device can be integrated with or attached to a catheter.

A method of performing ultrasound ablation with an ablation device having a longitudinal axis is disclosed. The method can include emitting, from a first emitter surface, a first ultrasound energy beam in a first beam first direction at a first beam first angle relative to the longitudinal axis. The method can include redirecting, via a first conical reflector, the first ultrasound energy beam.

Redirecting the first ultrasound energy beam can include redirecting the first ultrasound energy beam from the first beam first direction to a first beam second direction. The first beam second direction can extend at least partially in a radial direction away from the longitudinal axis at a first beam second angle relative to the longitudinal axis.

The first beam can extend at least partially around the longitudinal axis in the first beam first direction and in the first beam second direction.

The first beam first angle can be from 0 degrees to 90 degrees.

The first beam second angle can be from 0 degrees to 90 degrees.

The method can include moving the first conical reflector relative to the first emitter surface or vice versa or moving both the first conical reflector and the first emitter surface relative to one another.

The method can include moving the first conical reflector along the longitudinal axis relative to the first emitter surface or vice versa or moving both the first conical reflector and the first emitter surface long the longitudinal axis relative to one another.

The method can include emitting, from a second emitter surface, a second ultrasound energy beam in a second beam first direction at a second beam first angle relative to the longitudinal axis. The method can include redirecting, via a second conical reflector, the second ultrasound energy beam from the second beam first direction to a second beam second direction. The second beam second direction can extend at least partially in a radial direction away from the longitudinal axis at a second beam second angle relative to the longitudinal axis.

The second beam can extend at least partially around the longitudinal axis in the second beam first direction and the second beam second direction.

The second beam first angle can be from 0 degrees to 90 degrees.

The second beam second angle can be from 0 degrees to 90 degrees.

The second beam first direction can be opposite the first beam first direction.

The first beam can extend toward a focal zone in the first beam second direction. The second beam can extend toward the focal zone in the second beam second direction. At least a portion of the first and second beams can intersect in the focal zone.

The first beam can be at a first beam energy level. The second beam can be at a second beam energy level. The first and second beam energy levels can each alone be insufficient to ablate tissue. The first and second beam energy levels can be combined in the focal zone such that the combination of the first and second beam energy levels is sufficient to ablate tissue.

The method can include moving the focal zone from a first focal zone to a second focal zone.

Moving the focal zone from the first focal zone to the second focal zone can include at least one of moving the first conical reflector relative to at least one of the first emitter surface, the second emitter surface, and the second conical reflector; moving the second conical reflector relative to at least one of the first emitter surface, the second emitter surface, and the first conical reflector; moving the first emitter surface relative to at least one of the second emitter surface, the first conical reflector, and the second conical reflector; and

moving the second emitter surface relative to at least one of the first emitter surface, the first conical reflector, and the second conical reflector.

Moving the focal zone from the first focal zone to the second focal zone can include at least one of moving the first conical reflector relative to at least one of the first emitter surface, the second emitter surface, and the second conical reflector along the longitudinal axis; moving the second conical reflector relative to at least one of the first emitter surface, the second emitter surface, and the first conical reflector along the longitudinal axis; moving the first emitter surface relative to at least one of the second emitter surface, the first conical reflector, and the second conical reflector along the longitudinal axis; and moving the second emitter surface relative to at least one of the first emitter surface, the first conical reflector, and the second conical reflector along the longitudinal axis.

The focal zone can have an adjustable focal length from the longitudinal axis.

The focal length can be adjustable from 2 mm to 6 mm from the longitudinal axis.

The focal zone can have a toroid shape or a ring shape. The toroid shape or the ring shape can extend at least partially around the longitudinal axis.

### BRIEF SUMMARY OF THE DRAWINGS

The drawings shown and described are exemplary embodiments and non-limiting. Like reference numerals indicate identical or functionally equivalent features throughout.

FIG. 1A illustrates a variation of an ultrasound catheter system for the ablation of tissue.

FIG. 1B is a variation of a longitudinal cross-sectional view of the system of FIG. 1A taken along line 1B-1B.

FIG. 1C is a magnified view of the ultrasound device of FIG. 1A at section 1C-1C.

FIG. 1D is a variation of a magnified longitudinal cross-sectional view of the ultrasound device of FIG. 1C taken along line 1D-1D.

FIG. 1E is a magnified view of the system of FIG. 1B at section 1E-1E.

FIG. 1F illustrates the ultrasound device of FIG. 1C without the shield.

FIG. 1G illustrates the ultrasound device of FIG. 1D without the shield.

FIG. 1H illustrates a transparent view of the device of FIG. 1C.

FIG. 1I is a variation of a longitudinal cross-sectional view of the of the ultrasound device of FIG. 1H taken along line 1I-1I.

FIG. 1J is a schematic of the system of FIG. 1B with a guidewire.

FIG. 1K is a magnified view of the system of FIG. 1J at section 1K-1K.

FIG. 1L is a magnified view of the system of FIG. 1J at section 1L-1L.

FIG. 1M illustrates a transparent view of the system of FIG. 1A with a guidewire.

FIG. 1N illustrates a variation of a transverse cross-sectional view of the device of FIG. 1M taken along line 1N-1N.

FIG. 1O illustrates a variation of a transverse cross-sectional view of the device of FIG. 1M taken along line 1O-1O.

FIG. 1P illustrates a variation of a transverse cross-sectional view of the device of FIG. 1M taken along line 1P-LP.

FIG. 2A illustrates a variation of an ultrasound catheter system for the ablation of tissue.

FIG. 2B is a variation of a longitudinal cross-sectional view of FIG. 2A taken along line 2B-2B.

FIG. 3 illustrates a variation of an ultrasound device for the ablation of tissue.

FIG. 4A illustrates a longitudinal cross-section of a variation of an ultrasound system for the ablation of tissue having an adjustable reflector.

FIG. 4B is a variation of a top view of the adjustable reflector of FIG. 4A taken along line 4B-4B.

FIG. 4C illustrates a longitudinal cross-section of a variation of an ultrasound system for the ablation of tissue having multiple adjustable reflectors.

FIG. 4D is a variation of a top view of the multiple adjustable reflectors of FIG. 4C taken along line 4D-4D.

FIG. 5A illustrates a variation of a spacer for an ultrasound device.

FIG. 5B is a variation of a longitudinal cross-section of the spacer of FIG. 5A taken along line 5B-5B.

FIG. 5C illustrates a variation of a spacer for an ultrasound device.

FIG. 5D is a variation of a longitudinal cross-section of the spacer of figure SC taken along line 5D-5D.

FIG. 5E is a variation of a transverse cross-section of the spacer of FIGS. 5A and 5C taken along line 5E-5E.

FIG. 5F is another variation of a transverse cross-section of the spacer of FIGS. 5A and 5C taken along line 5E-5E.

FIG. 6A illustrates a variation of an ultrasound catheter system for the ablation of tissue.

FIG. 6B is a variation of a longitudinal cross-sectional view of the system of FIG. 6A taken along line 6B-6B.

FIG. 6C is a magnified view of the system of FIG. 6B at section 6C-6C.

FIGS. 7A1 and 7A2 illustrate side and top views of a variation of a transducer.

FIGS. 7B1 and 7B2 illustrate side and top views a variation of a transducer.

FIGS. 7C1 and 7C2 illustrate side and top views a variation of a transducer.

FIGS. 7D1 and 7D2 illustrate side and top views a variation of a transducer.

FIGS. 7E1 and 7E2 illustrate side and top views a variation of a transducer.

FIGS. 7F1 and 7F2 illustrate side and top views a variation of a reflector.

FIG. 8A is a schematic illustration of the ultrasound device of FIGS. 1A-1P emitting energy.

FIG. 8B is a schematic illustration of various aspects of the ultrasound device of FIGS. 1A-1P emitting energy.

FIG. 9 illustrates the ultrasound catheter system of FIGS. 1A-1P emitting energy in a blood vessel.

FIGS. 10A1-10D2 are various schematic illustrations of ultrasound catheter systems emitting energy in a blood vessel.

FIGS. 11A and 11B illustrate perspective views of the ultrasound catheter system of FIGS. 2A and 2B emitting energy.

FIGS. 11C and 11D illustrate side views of the ultrasound catheter system of FIGS. 2A and 2B emitting energy.

FIG. 12 illustrates the ultrasound device of FIG. 3 emitting energy.

FIGS. 13A and 13B illustrate the ultrasound system of FIGS. 4A and 4B emitting energy.

FIGS. 13C and 13D illustrate the ultrasound system of FIGS. 4C and 4D emitting energy.

FIGS. 14A1 and 14A2 illustrate the transducer of FIGS. 7A1 and 7A2 emitting energy.

FIGS. 14B1 and 14B2 illustrate the transducer of FIGS. 7B1 and 7B2 emitting energy.

FIGS. 14C1 and 14C2 illustrate the transducer of FIGS. 7C1 and 7C2 emitting energy.

FIGS. 14D1 and 14D2 illustrate the transducer of FIGS. 7D1 and 7D2 emitting energy.

FIGS. 14E1 and 14E2 illustrate the transducer of FIGS. 7E1 and 7E2 emitting energy.

FIGS. 14F1 and 14F2 illustrate the reflector of FIGS. 7F1 and 7F2 redirecting energy.

### DETAILED DESCRIPTION

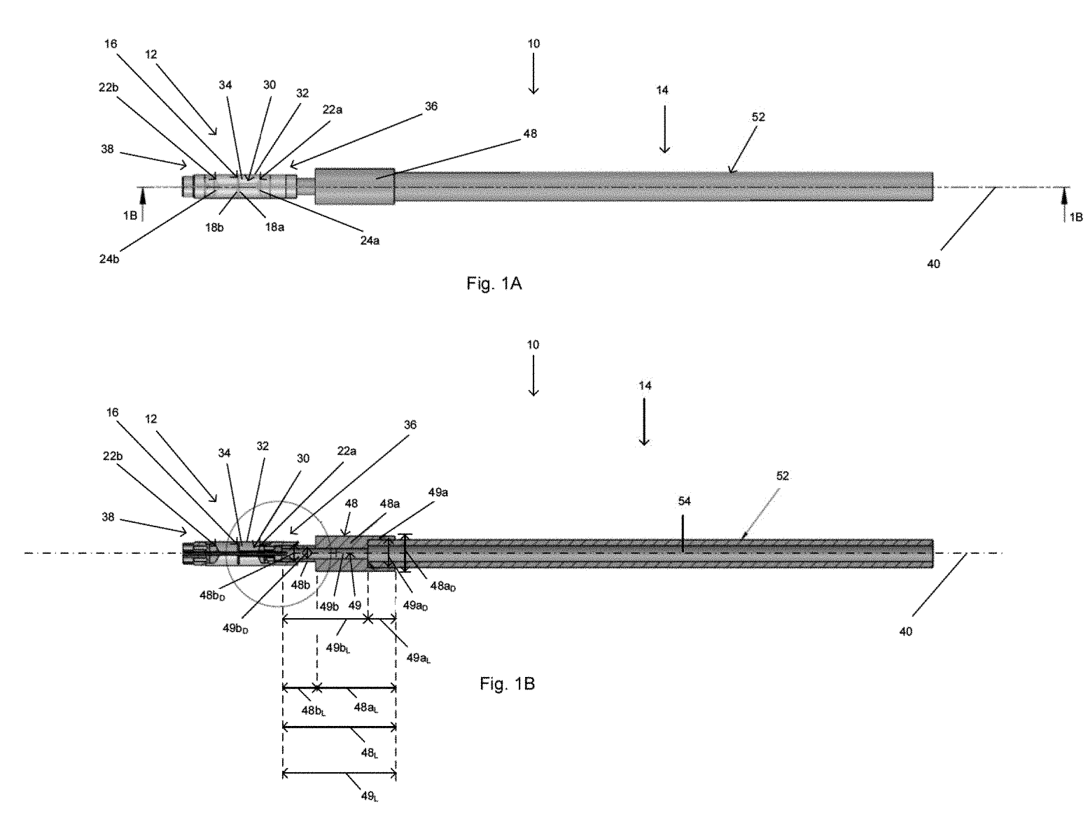

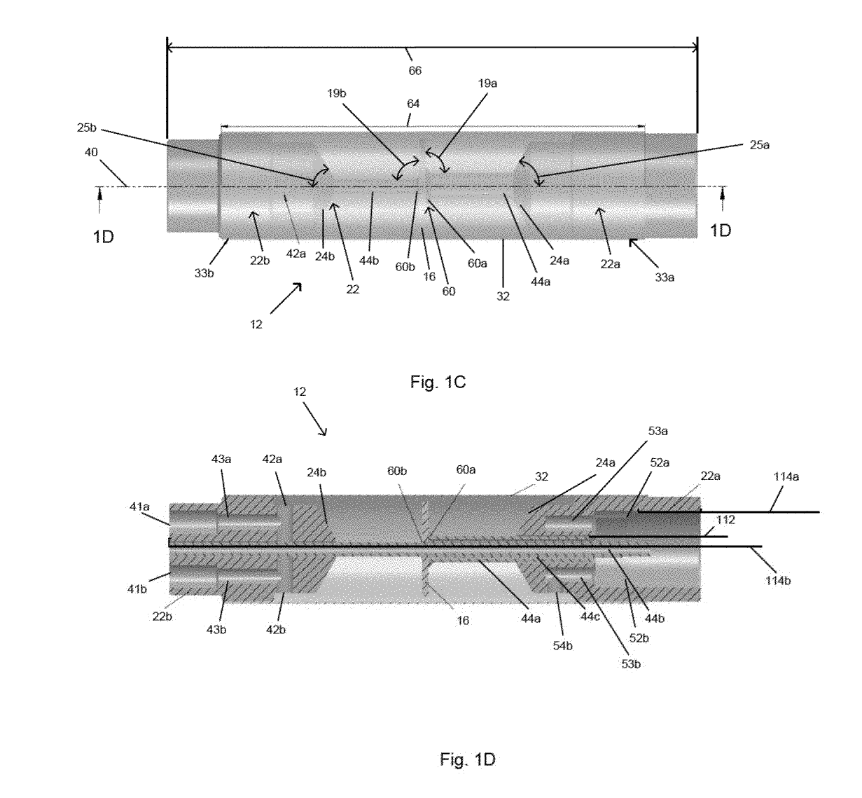

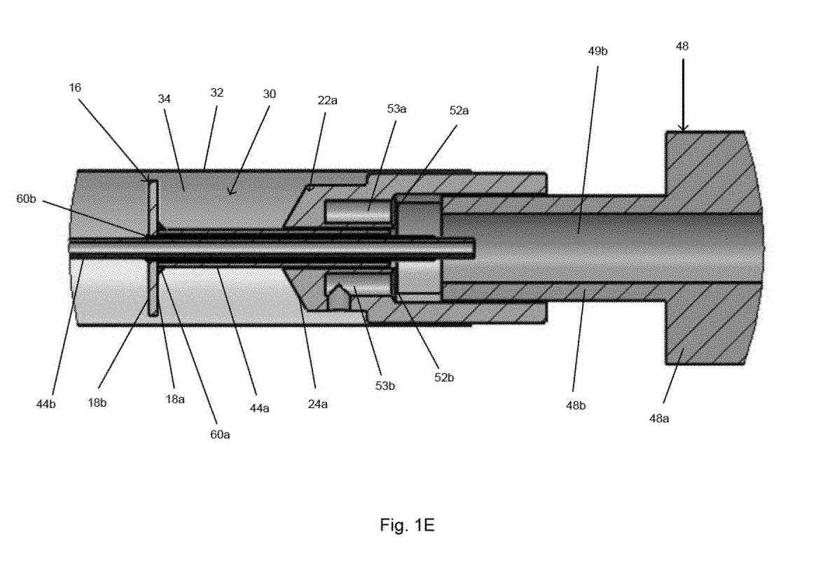

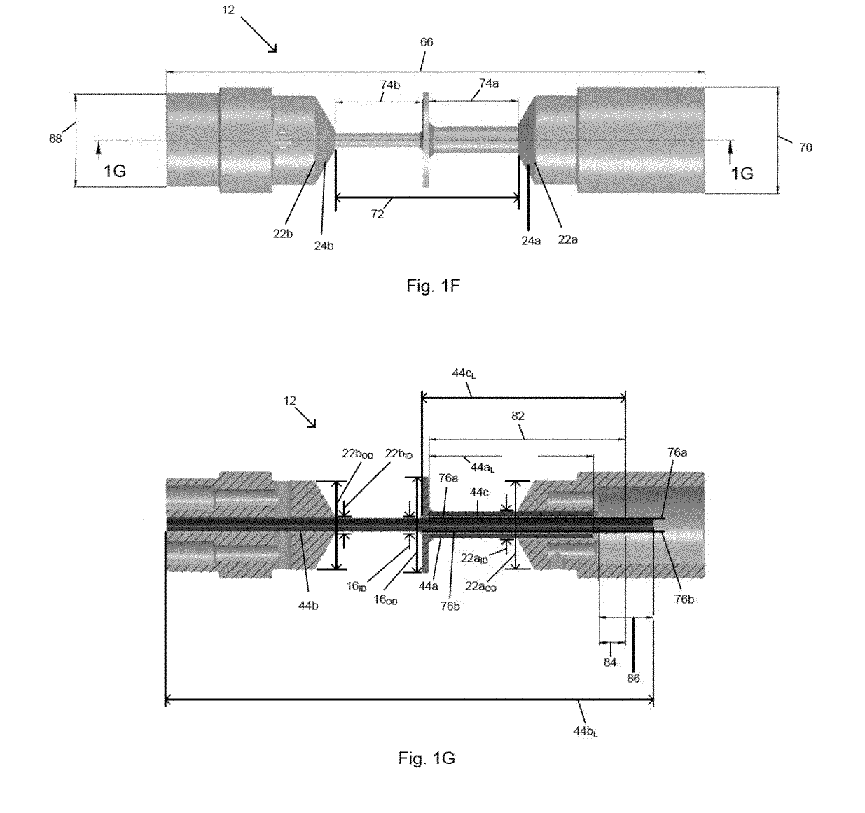

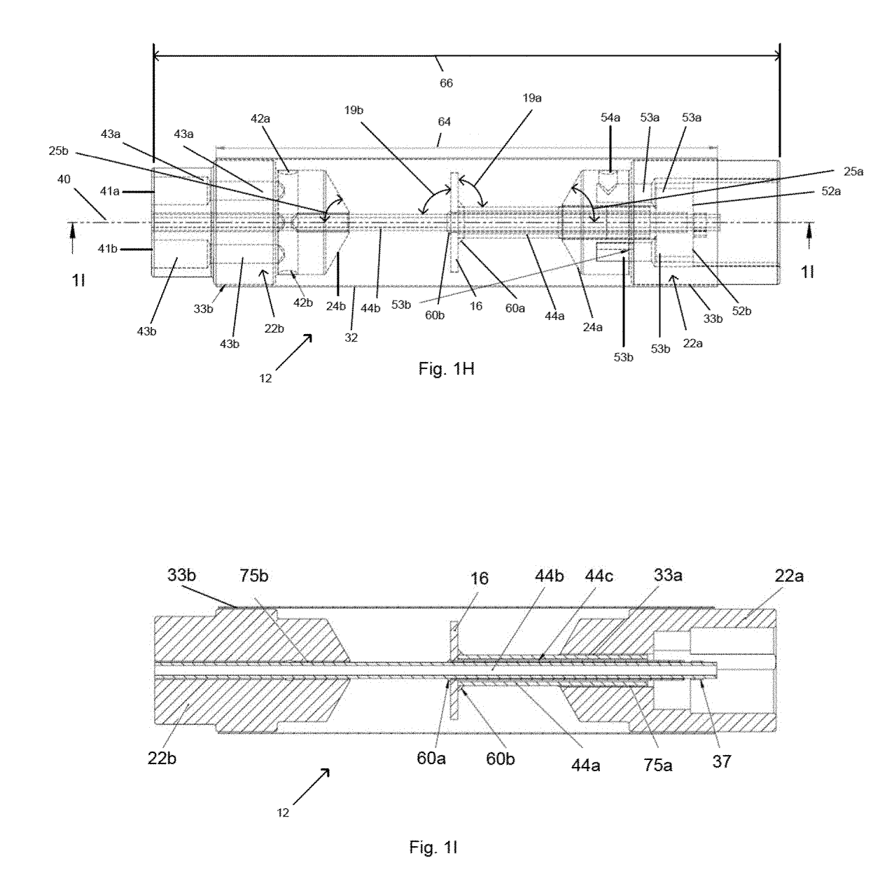

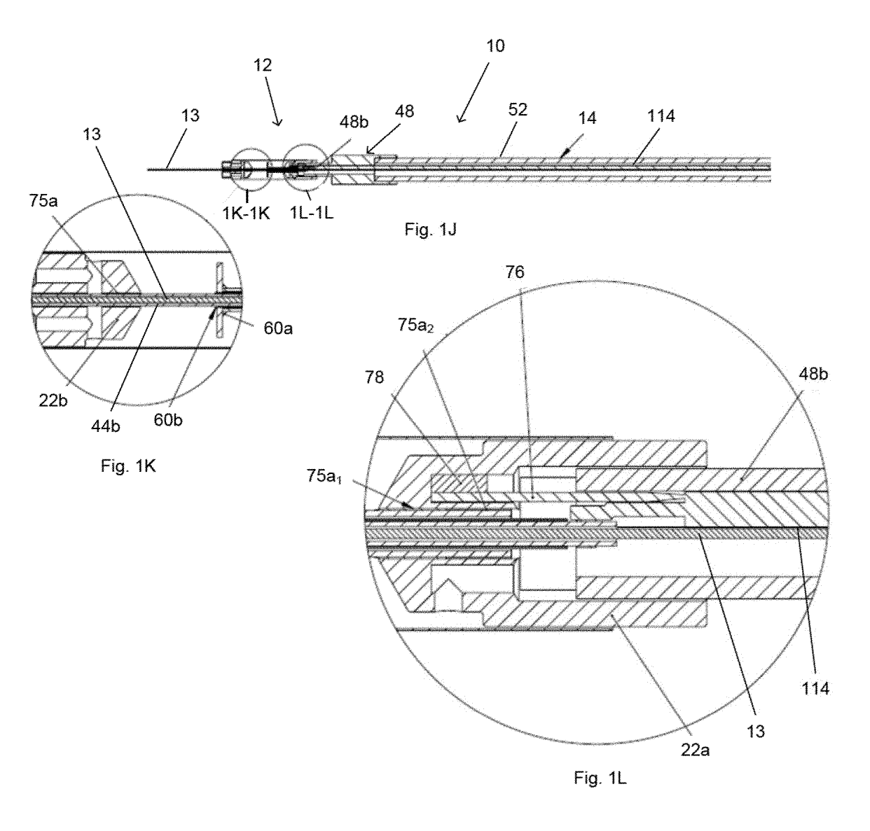

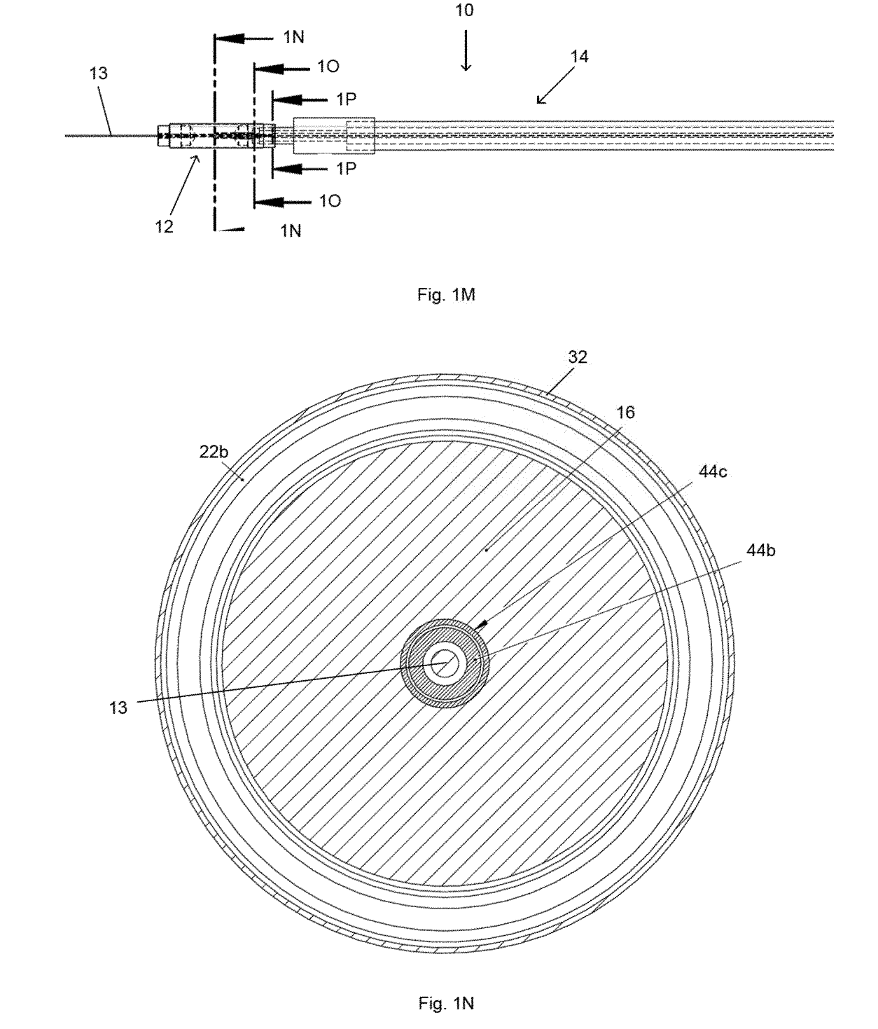

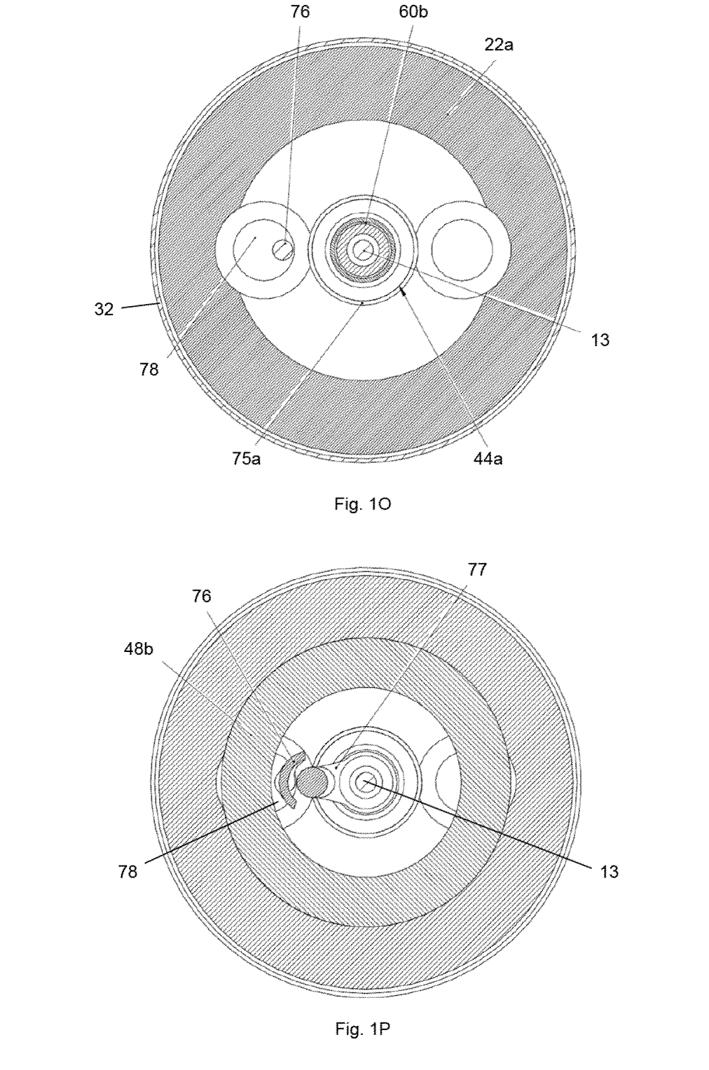

FIGS. 1A-1P illustrate a variation of an ultrasound catheter system 10 for the ablation of tissue. FIG. 1A illustrates that the system 10 can have a catheter 14 and one or more ultrasound devices 12. The ultrasound device(s) 12 can be directly or indirectly attached to the catheter 14. For example, the ultrasound device(s) 12 can be attached to the catheter via a coupler 48. The ultrasound device(s) 12 can be integrated with the catheter 14. For example, the ultrasound device(s) 12 can be integrated with a catheter shaft 52 of the catheter 14. The catheter 14 can be an over-the-wire catheter.

The ultrasound device 12 can be adjustable or non-adjustable. The ultrasound device can deliver ultrasound energy to body tissue to an adjustable or fixed depth. The depth of the energy delivery can be adjusted. For example, the depth of the energy delivered can be adjusted during use.

The system 10 can be navigated through a vasculature such that the ultrasound device 12 is positioned in a vein or artery. The device 12 can be delivered via a guidewire. The ultrasound device 12 can be positioned at one or more ablation target sites in a vein or artery. The artery can be, for example, a left or right renal artery. When positioned in a left or right renal artery, the ultrasound device 12 can be used to perform renal denervation with ultrasound energy. Such ultrasound treatment can treat hypertension, including hypertension resistant to other treatments.

The ultrasound device 12 can selectively target afferent nerves that innervate blood vessels. For example, the ultrasound device 12 can target afferent nerves outside of a blood vessel wall, such as the afferent nerves in and around the adventitia of the blood vessel. For example, the ultrasound device 12 can selectively target afferent nerves that arborize around a renal artery.

FIG. 1A illustrates that the system 10 can have a longitudinal axis 40 and that the ultrasound device 12 can have a first longitudinal end 36 and a second longitudinal end 38. As shown, the ultrasound device 12 can be at a terminal end of the catheter 14. However, the ultrasound device 12 can be attached to or integrated with the catheter 14 anywhere along the length of the catheter 14, for example, anywhere between a first and second terminal end of the catheter 14.

The device 12 can have one or more transducers 16 and one or more reflectors 22. For example, FIG. 1A illustrates that the ultrasound device 12 can have a transducer 16, a first reflector 22a, and a second reflector 22b.

The transducer 16 can be a self-vibrating transducer, for example, a piezoelectric transducer. The self-vibrating transducer 16 can be connected to a power source via one or more wires and/or can be wirelessly powered. The transducer 16 can be connected to an ultrasound energy source, for example, a tube configured to vibrate when activated. The tube can transmit ultrasonic vibrations to the transducer 16, which can, in turn, vibrate the transducer 16. The tube can be, for example, a hypotube. The tube can be connected to a power source, wired and/or wirelessly.

The transducer 16 can emit ultrasound energy when activated. The transducer 16 can have a harmonic frequency range from about 9 MHz to about 15 MHz. For example, the transducer 16 can emit ultrasound energy at a frequency from about 9 MHz to about 15 MHz, from about 10 MHz to about 14 MHz and/or from about 11 MHz to about 13 Mhz. The ultrasound energy level can be adjusted. For example, the ultrasound energy level can be adjusted during use.

The transducer 16 can emit the same or different ultrasound energy level toward each of the one or more reflectors 22. For example, the transducer 16 can emit the same or different ultrasonic frequency toward the first and/or second reflectors 22a, 22b.

The transducer(s) 16 can be a disc or an annular disc. The distal diameter 68 can be from about 0.050 inches to about 0.150 inches (e.g., about 0.088 inches). The transducer(s) 16 can have one or more holes, for example, to allow one or more shafts and/or wires to pass through (e.g., from one side of the transducer to another side of the transducer). A hole can be in the center of the transducer(s) 16. The hole can be centered with the longitudinal axis 40. The hole can be offset from the longitudinal axis 40. The hole can have a curved or polygonal cross-section (e.g., circular, triangular, square).

The one or more transducers 16 can each have one or more emitter surfaces 18. FIG. 1A illustrates that the transducer 16 can have a first emitter surface 18a and a second emitter surface 18b. The number of emitter surfaces can be, for example, 1 to 10 or more (e.g., 500 or less emitter surfaces).

The first emitter surface 18a can have one or more planar emitter faces and/or one or more curved emitter faces. The first emitter surface 18a can be at least partially curved and/or flat. At least a portion of the first emitter surface 18a can be polygonal; for example, the first emitter surface 18a can have two or more planar emitter faces angled relative to one another. The angle between the two or more planar emitter faces of the first emitter surface 18a can be fixed or adjustable. At least a portion of the first emitter surface 18a can be concave and/or convex.

The second emitter surface 18b can have one or more planar emitter faces and/or one or more curved emitter faces. The second emitter surface 18b can be at least partially curved and/or flat. At least a portion of the second emitter surface 18b can be polygonal; for example, the second emitter surface 18b can have two or more planar emitter faces angled relative to one another. The angle between the two or more planar emitter faces of the second emitter surface 18b can be fixed or adjustable. At least a portion of the second emitter surface 18b can be concave and/or convex.

For example, FIG. 1A illustrates that the first and second emitter surfaces 18a, 18b can both be flat. The first and second emitter surfaces 18a, 18b can be parallel or angled relative to one another. For example, FIG. 1A illustrates that the first and second emitter surfaces 18a, 18b can be parallel to each other.

The one or more emitter surfaces 18 can be at one or more emitter surface angles 19 relative to the longitudinal axis 40. FIG. 1A illustrates that the first emitter surface 18a can be at a first emitter surface angle 19a (see FIG. 1C) relative to the longitudinal axis 40. The first emitter surface angle 19a can be from about 0 degrees to about 90 degrees, for example, about 90 degrees. The second emitter surface 18b can be at a second emitter surface angle 19b (see FIG. 1C) relative to the longitudinal axis 40. The second emitter surface angle 19b can be from about 0 degrees to about 90 degrees, for example, about 90 degrees. An axis normal to the first emitter surface 18a can extend at least partially in a first longitudinal direction toward the first longitudinal end 36 of the device 12 and an axis normal to the second emitter surface 18b can extend at least partially in a second longitudinal direction toward the second longitudinal end 38 of the device 12. FIG. 1A illustrates that the first and second emitter surfaces 18a, 18b can face opposite directions from one another.

FIG. 1A illustrates that the one or more reflectors 22 can have a tapered three-dimensional shape. The tapered three-dimensional shape can have one or more tapered faces 24 (also referred to as reflective surfaces) that extend between a base plane and an apex. For example, the tapered three-dimensional shape can be a cone or a polyhedron. The cone can be a regular or oblique cone. The polyhedron can be a pyramid, for example, with three or more tapered faces between the base plane and the apex. The polyhedron can be a regular or oblique polyhedron.

The tapered three-dimensional shape can have one or more tapered faces that extend between a first plane and a second plane. For example, the tapered three-dimensional shape can be a frustum. The frustum can be a right or oblique frustum. The frustum can be a conical frustum or a polyhedral frustum. The polyhedral frustum can be a pyramidal frustum. The two planes defining the frustum can be parallel or angled relative to one another.

The tapered three-dimensional shape can have a center axis. The tapered three-dimensional shape can extend partially or completely around the center axis. The center axis can be parallel or angled relative to the device longitudinal axis 40. The center axis can coincide with or be offset from the device longitudinal axis 40. For example, FIG. 1A illustrates that the center axes of the reflectors 22a, 22b can coincide with the device longitudinal axis 40. The tapered three-dimensional shape can extend at least partially (e.g., partially or completely) around the longitudinal axis 40, symmetrically or asymmetrically. The tapered faces 24 can be angled relative to the longitudinal axis 40, for example, from about 15 degrees to about 75 degrees (e.g., about 45 degrees). The tapered faces can be angled relative to the base plane of the cone or polyhedron, for example, from about 15 degrees to about 75 degrees (e.g., about 45 degrees). As shown, the tapered faces can each extend at least partially in a longitudinal direction and at least partially in a radial direction away from the longitudinal axis 40.

The one or more reflectors 22 can have a conical shape, including conical, truncated-conical, or frusto-conical. The one or more reflectors 22 can have a polyhedral shape, including polyhedral, truncated-polyhedral, or frusto-polyhedral. The one or more reflectors 22 can have a pyramidal shape, including pyramidal truncated-pyramidal, or frusto-pyramidal. The one or more reflectors 22 can have the same or different shapes relative to one another. FIG. 1A illustrates that the first and second reflectors 22a, 22b can have a frusto-conical shape; however, as described above, any cone, polyhedron, or frustum shape is appreciated, including polyhedrons and/or frustums having three or more tapered faces (e.g., 25 faces or less, 50 faces or less, 100 faces or less, 150 faces or less, 250 faces or less, or 500 faces or less). The first and second reflectors 22a, 22b can function like or otherwise approximate the shape of a frusto-conical reflector when they have a polyhedral frustum shape with, for example, 10 or more faces. As described above, the first and second reflectors 22a, 22b can be right and/or oblique tapered three-dimensional shapes, for example, right and/or oblique cones, polyhedrons, and/or frustums. As shown, the first and second reflectors 22a, 22b can extend at least partially (e.g., partially or completely) around the longitudinal axis 40.

The first and second reflectors 22a, 22b can each have one or more reflective surfaces 24. FIG. 1A illustrates that the first and second reflectors 22a, 22b can have first and second reflective surfaces 24a, 24b, respectively. As described above, the first and second reflective surfaces 24a, 24b can have a conical, truncated-conical, or a frusto-conical shape. The first reflective surface 24a can be at a first reflector surface angle 25a (see FIG. 1C) relative to the longitudinal axis 40 and the second reflective surface 24b can be at a second reflector surface angle 25b (see FIG. 1C) relative to the longitudinal axis 40. The first reflector surface angle 25a can be from about 15 degrees to about 75 degrees, for example, about 45 degrees. The second reflector surface angle 25b can be from about 15 degrees to about 75 degrees, for example, about 45 degrees. The first reflective surface 24a can extend at least partially around the longitudinal axis 40. The second reflective surface 24b can extend at least partially around the longitudinal axis 40.

FIG. 1A illustrates that the first reflector 22a can be at a first end of the device 12, for example, at a first longitudinal end 36 of the device 12. The second reflector 22b can be at a second end of the device 12, for example, at a second longitudinal end 38 of the device 12. The first and second reflectors 22a, 22b can be proximal and distal reflectors, respectively. The transducer 16 can be between the first and second reflectors 22a, 22b. However, the transducer(s) and reflector(s) 16, 22 can be on different axes relative to one another.

An axis (e.g., center axis) of the transducer(s) and reflector(s) 16, 22 can coincide with, be offset from, or be angled relative to the longitudinal axis 40 of the device 12. For example, FIG. 1A illustrates that the center axes of the transducer 16 and the first and second reflectors 22a, 22b can coincide with the longitudinal axis 40 of the device 12.

The one or more reflectors 22 (e.g., first and second reflectors 22a, 22b) can be a highly polished material (e.g., steel). The one or more tapered surfaces 24 (e.g., first and second reflector surfaces 24a, 24b) can be a highly polished material (e.g., steel). The one or more reflectors can be a rigid and/or semi-rigid material.

The one or more reflectors can have one or more secondary focusing features (e.g., lens, apertures, aperture windows, or combinations thereof). The one or more reflectors can have one or multiple segments. The one or more reflectors can have one or multiple reflective segments. The device 12 can have one or more redirected reflectors. The one or more redirected reflectors can be adjustable. The redirected reflectors can redirect energy from the transducer and or from another reflector (e.g., from the first and/or second reflectors 22a, 22b and/or from another redirected reflector).

FIG. 1A illustrates that the ultrasound device 12 can have a shield 32. The shield 32 can extend from the first longitudinal end 36 to the second longitudinal end 38 of the device 12. The shield 32 can extend at least partially around the device 12. The shield 32 can extend at least partially around the one or more transducers 16 and reflectors 22. At least a portion of the shield 32 can be transparent such that less than 10% of ultrasound energy that passes therethrough is attenuated. The shield 32 can be a tube. The shield can have a cylindrical shape. The shield can have a curved or polygonal cross-section (e.g., circular, triangular, square). At least a portion of the shield 32 can be directly or indirectly attached to the first and second longitudinal ends 36, 38 of the device 12. The shield 32 can be attached to the first and second longitudinal ends 36, 38 with glue, thermal bonding, adhesive (e.g., LOCTITE 4307). For example, the shield 32 can be directly or indirectly attached to a portion of the reflectors 22 at shield first bond 33a and at shield second bond 33b. For example, the shield 32 can be attached to a side or base portion of the reflectors 22. The one or more reflectors 22 can be moveable relative to the shield 32. For example, the first and/or second reflectors 22a, 22b can be translated along the longitudinal axis 40 relative to the shield 32 and/or the transducer 16. The shield can be made of polymethylpentene.

FIG. 1A illustrates that the ultrasound device 12 can have a chamber 30. The shield 32 can define at least a portion of the chamber 30. The one or more transducers (e.g., transducer 16) and reflectors (e.g., reflectors 22a, 22b) can be in the chamber 30. An ultrasound coupling fluid 34 can be in the chamber 30. The ultrasound coupling fluid 34 can be, for example, saline. The coupling fluid 34 can provide acoustic coupling for the ultrasound energy emitted from the one or more transducers 16. In lieu of or in addition to the shield 32, the one or more transducers can be coated with polymethylpentene, for example, a polymethylpentene film. The polymethylpentene material (e.g., coating, film) can provide acoustic coupling for the ultrasound energy emitted from the transducer(s) and protect the emission surfaces of the transducer(s). The chamber 30 can have a volume from about 2 mL to about 20 mL (e.g., about 5 mL). The chamber 30 can have a length from about 0.050 inches to about 0.700 inches (e.g., about 0.350 inches). The size (e.g., volume and/or length) of the chamber 30 can be fixed. The size (e.g., volume and/or length) of the chamber 30 can be adjusted, for example, from one or more first sizes to one or more second sizes. For example, the volume of the chamber 30 can be adjusted from a first volume to a second volume smaller than the first volume and/or to a third volume larger than the first volume. The size of the chamber 30 can be adjusted, for example, by moving the first and/or second reflectors 22a, 22b toward or away from the first and/or second longitudinal ends 36, 38 of the device 12. The size of the chamber 30 can be adjusted, for example, by moving the first longitudinal end 36 of the device 12 toward or away from the second longitudinal ends 38 of the device 12 and/or vice versa.

FIG. 1B illustrates that the coupler 48 can have a coupler channel 49. FIG. 1B illustrates that the coupler 48 can have a coupler first portion 48a having a coupler first channel 49a having a first channel diameter 49a_{D} and a coupler second portion 48b having a coupler second channel 49b having a second channel diameter 49b_{D} the same or different from the first channel diameter 49a_{D}. As shown, the coupler second channel 49b can extend at least partially into the coupler first portion 48a. The coupler first portion 48a can be at a first (e.g., proximal) end of the coupler 48 and the coupler second portion 48b can be at a second (e.g., distal) end of the coupler 48. The coupler first portion 48a can be directly or indirectly attached to the catheter shaft 52, for example, to a distal end of the catheter shaft 52. The coupler second portion 48b can be directly or indirectly attached to the ultrasound device 12, for example, to the first longitudinal end 36 of the device 12. The distal end of the catheter shaft 52 can be in the coupler first channel 49a and/or fit around the coupler first portion 48a. The distal end of the catheter shaft 52 can be attached to the coupler first portion 48a with glue, adhesive (e.g., LOCTITE 4310), a friction fit, and/or with a screw fit. The first longitudinal end 36 of the device can be in the coupler second channel 49b and/or fit around the coupler second portion 48b. The first longitudinal end 36 of the device attached to the coupler second portion 48b glue, adhesive (e.g., LOCTITE 4310), a friction fit, and/or with a screw fit. One or more controls and/or wires (not shown) can extend through the catheter shaft 52 and/or coupler channel 49 (e.g., coupler first and second channels 49a, 49b). The one or more controls and/or wires can be directly or indirectly connected to one or more features of the device 12, for example, the transducer(s) 16 and/or the one or more reflectors 22 (e.g., first and second reflectors 22a, 22b).

FIG. 1B illustrates that the coupler 48 can have a coupler length 48_{L} from about 0.200 inches to about 1.000 inches (e.g., about 0.700 inches). The coupler first portion 48a can have a coupler first portion length 48a_{L} from about 0.100 inches to about 0.900 inches (e.g., about 0.500 inches). The coupler second portion 48b can have a coupler second portion length 48b_{L} from about 0.100 inches to about 0.900 inches (e.g., about 0.200 inches). The coupler first portion 48a can have a coupler first portion diameter 48a_{D} from about 0.030 inches to about 0.200 inches (e.g., about 0.110 inches). The coupler second portion 48b can have a coupler second portion diameter 48b_{D} from about 0.030 inches to about 0.200 inches (e.g., about 0.070 inches). The coupler channel 49 can have a coupler channel length 49_{L} from about 0.200 inches to about 1.000 inches (e.g., about 0.700 inches). The coupler first channel 49a can have a coupler first channel length 49a_{L} from about 0.200 inches to about 0.900 inches (e.g., about 0.150 inches). The coupler second channel 49b can have a coupler second channel length 49b_{L} from about 0.200 inches to about 0.900 inches (e.g., about 0.550 inches). The first channel diameter 49a_{D} can be from about 0.030 inches to about 0.200 inches (e.g., about 0.100 inches). The second channel diameter 49b_{D} can be from about 0.030 inches to about 0.200 inches (e.g., about 0.060 inches).

FIG. 1C illustrates that the device 12 can have a device length 66 and a shield length 64. The device length 66 can be from about 0.200 inches to about 0.800 inches (e.g., about 0.507 inches). The device length 66 can be fixed. The device length 66 can be adjustable. For example, the device length 66 can be increased and/or decreased. The device 12 can have one or more device configurations having one or more corresponding device lengths to one or more other device configurations having one or more other corresponding device lengths. The device length 66 can be increased and/or decreased, for example, from a device first length (associated with a device first configuration) to a device second length (associated with a device second configuration). The device 12 can be adjusted from a device first configuration having a device first length to a device second configuration having a device second length greater than or less than the device first length. The device length 66 can be increased, for example, from a fully contracted first configuration, from a partially contracted first configuration, and/or from a partially expanded first configuration having a device first length to a device second configuration having a device second length greater than the device first length. The device length 66 can be decreased, for example, from a fully expanded first configuration, from a partially expanded first configuration, and/or from a partially contracted first configuration having a device first length to a device second configuration having a device second length less than the device first length. The device length 66 can be increased and/or decreased, for example, from a neutral configuration (e.g., a fully contracted configuration, a partially contracted configuration, a configuration with a device length 66 halfway between a fully expanded and fully contracted configuration, a partially expanded configuration, and/or a fully expanded configuration). For example, the device length 66 can be about 0.507 inches when the device 12 is in a first (e.g., partially expanded or fully expanded) configuration and can be less than about 0.507 inches when the device 12 is in a second (e.g., partially contracted or fully contracted) configuration. The shield length 64 can be from about 0.100 inches to about 0.700 inches (e.g., about 0.405 inches).

The device 12 can have one or more shafts 44 (e.g., 1, 2, 3 or more shafts). One or more of the shafts 44 can be telescoping shafts, e.g., relative to each other and % or relative to another space/channel (e.g., a reflector channel, a coupler channel). FIG. 1C illustrates that the device 12 can have a first shaft 44a and a second shaft 44b. The first shaft 44a can telescope relative to the second shaft 44b and/or vice versa. The one or more reflectors 22 can be directly or indirectly attached to the one or multiple shafts 44. The first reflector 22a can be directly or indirectly attached to the first shaft 44a. The second reflector 22b can be directly or indirectly attached to the second shaft 44b. The transducer 16 can be attached to the one or more shafts 44 with one or more bonds 60. A first side of the transducer 16 (e.g., the first emitter surface 18a) can be directly or indirectly attached to the first shaft 44a, for example, with a first bond 60a. The first bond 60a can be an annular bond. The first bond 60a can be formed with epoxy, for example, conductive epoxy. A second side of the transducer 16 (e.g., the second emitter surface 18b) can be directly or indirectly attached to the second shaft 44b, for example, with a second bond 60b. The second bond 60b can be an annular bond. The second bond 60bb can be formed with epoxy, for example, conductive epoxy. The first and second shafts 44a, 44b can be hypotubes.

As described above, the first and second shafts 44a, 44b can be moveable relative to one another. For example, the first or second shaft 44a, 44b can be slideable within or over the second or first shaft 44b, 44a, respectively, and/or vice versa. This movability can advantageously lengthen or shorten the device length 66, which can in turn adjust the distances between the one or more reflectors 22 (e.g., first and second reflectors 22a, 22b) relative to each other and/or the distances between the one or more reflectors 22 (e.g., first and second reflectors 22a, 22b) relative to the one or more transducers 16.

One or more of the reflectors 22 can be moveable relative to the longitudinal axis 40 and/or to each other. For example, the first and/or second reflector 22a, 22b can be translated and/or rotated relative to the longitudinal axis 40. The first or second reflector 22a, 22b can be translated and/or rotated relative to the second or first reflector 22b, 22a, respectively, and/or vice versa. One or more of the reflectors 22 can be moveable relative to the longitudinal axis 40 and/or to the one or more transducers 16. For example, the first and/or second reflector 22a, 22b can be translated and/or rotated relative to the transducer 16. The first and/or second reflector 22a, 22b can be translated and/or rotated relative to the first and/or second emitter surfaces 18a, 18b. This adjustability can advantageously allow the ultrasound device 12 to delivery ultrasound energy to tissue at adjustable treatment depth. For example, the treatment depth can be greater (e.g., farther from a vessel wall) when the first and second reflectors 22a, 22b are farther apart than when closer together.

The ultrasound device 12 can have one or multiple coupling fluid ports. The ultrasound device 12 can have one or multiple coupling fluid channels. Each of the one or multiple coupling fluid ports and/or channels can have one or multiple functions (e.g., inlet, outlet, recirculation, air evacuation). For example, the ultrasound device 12 can have one or more coupling fluid inlets and one or more coupling fluid outlets (also referred to as one or more coupling fluid inlet ports and one or more coupling fluid outlet ports) for the coupling fluid 34 to flow into and out of the coupling chamber 30. The ultrasound device 12 can have one or more coupling fluid inlet channels and one or more coupling fluid outlet channels for the coupling fluid 34 to flow into and out of the coupling chamber 30. The coupling fluid inlet(s) and coupling fluid inlet channel(s) can be in fluid communication with one another. The coupling fluid outlet(s) and coupling fluid outlet channels(s) can be in fluid communication with one another. The ultrasound device 12 can have one or more coupling fluid recirculation ports. The ultrasound device 12 can have one or more coupling fluid recirculation channels. The coupling fluid recirculation port(s) and coupling fluid recirculation channel(s) can be in fluid communication with one another. The flow of coupling fluid 34 into and out of the coupling chamber 30, via the coupling fluid inlets, outlets, recirculation ports, inlet channels, outlet channels, and/or recirculation channels, can advantageously keep the temperature of the device 12 within safe operable limits, for example, from about 5.0 degrees Celsius to about 42.0 degrees Celsius (e.g., about 5.0 degrees Celsius, about 37.0 degrees Celsius). The flow of coupling fluid 34 can function as cooling fluid that can keep the transducer 16 cool (e.g., about 30.0 degrees Celsius).

The ultrasound device 12 can have one or more inlets and one or more outlets to evacuate fluid (e.g., air) from the chamber 30 during and/or prior to use of the device 12. For example, a coupling fluid inlet, coupling fluid inlet channel, a coupling fluid outlet, and/or a coupling fluid outlet channel can be used to evacuate fluid (e.g., air) from the chamber 30 during and/or prior to use of the device 12. An air evacuation outlet (e.g., a dedicated air evacuation outlet) can be used to evacuate fluid (e.g., air) from the chamber 30 during and/or prior to use of the device 12. The air evacuation outlet can be in fluid communication with an air evacuation channel.

FIGS. 1C and 1D illustrate that the ultrasound device 12 can have one or more coupling fluid outlet ports 42 one or more coupling fluid inlet ports 52. FIG. 1D illustrates that the device 12 can have a coupling fluid first outlet port 42a in fluid communication with a coupling fluid first outlet channel 43a and that the device 12 can have a coupling fluid second outlet port 42b in fluid communication with a coupling fluid second outlet channel 43b. The coupling fluid first outlet port and channel 42a. 43a can be in fluid communication with a coupling fluid first outlet opening 41a and the coupling fluid second outlet port and channel 42b, 43b can be in fluid communication with a coupling fluid second outlet opening 41b. As shown, the coupling fluid first outlet port and channel 42a, 43a and the coupling fluid second outlet port and channel 42b, 43b can be in the second reflector 22b. The coupling fluid first and second channels 43a, 43b can have one or multiple channel diameters (e.g., the two shown in FIG. 1D).

FIG. 1D illustrates that the device 12 can have a coupling fluid first inlet port 52a in fluid communication with a coupling fluid first inlet channel 53a and that the device 12 can have a coupling fluid second inlet port 52b in fluid communication with a coupling fluid second inlet channel 53b. The coupling fluid first inlet port and channel 52a, 53a can be in fluid communication with a coupling fluid first inlet opening 54a (see FIG. 1H) and the coupling fluid second inlet port and channel 52b, 53b can be in fluid communication with a coupling fluid second inlet opening 54b. As shown, the coupling fluid first inlet port and channel 52a, 53a and the coupling fluid second inlet port and channel 52b, 53b can be in the first reflector 22a.

As described above, the coupling fluid first inlet and channel 52a, 53a and/or the coupling fluid second inlet and channel 52b, 53b can be, or can be used as, a fluid (e.g., air) evacuation outlet and channel during and/or prior to use of the device 12.

The coupling fluid first inlet and channel 52a, 53a and/or the coupling fluid second inlet and channel 52b, 53b can be, or can be used as, a coupling fluid recirculation port and channel during and % or prior to use of the device 12.

The device 12 can have, for example, two coupling fluid outlets and outlet channels, one coupling fluid inlet and inlet channel, and one coupling fluid recirculation port and recirculation channel.

The coupling fluid inlets, outlets, inlet channels, and/or outlet channels 42a, 42b, 43a, 43b, 52a, 52b, 53a, 53b can be in the coupling chamber 30. The coupling fluid inlets, outlets, inlet channels, and/or outlet channels 42a, 42b, 43a. 43b, 52a, 52b, 53a, 53b can be in fluid communication with the coupling chamber 30. The coupling fluid outlet ports and channels 42a, 42b, 43a, 43b can be in fluid communication with an outside environs, for example, a lumen of a blood vessel when the system 10 is in use (e.g., a lumen of a renal artery). The coupling fluid inlet ports and channels 52a, 52b, 53a, 53b can be in fluid communication with a coupling fluid source. The coupling fluid recirculation port (e.g., the first and/or second inlet ports 52a, 52b and/or the first and/or second inlet channels 53a, 53b) can be in fluid communication with a recirculation reservoir and/or with the coupling fluid source. The fluid (e.g., air) evacuation ports and channels (e.g., coupling fluid first inlet and channel 52a, 53a and/or the coupling fluid second inlet and channel 52b, 53b) can be in fluid communication with a negative pressure source.

Coupling fluid 34 can be delivered to the chamber 30 via the one or more coupling fluid inlets 52 (e.g., coupling fluid first and second inlets 52a, 52b). Coupling fluid 34 can be jettisoned (also referred to as dumped or circulated) into a blood vessel via the one or more coupling fluid outlets 42 (e.g., coupling fluid first and second outlets 42a, 42b). Coupling fluid 34 can be recycled into the catheter via one or more coupling fluid recirculation ports (e.g., as described above). Fluid (e.g., air) can be evacuated from the chamber 30 by, for example, applying a negative pressure to the chamber 30 to force out undesirable fluid (e.g., air) from the chamber 30.

The system 10 can have one or more transducer controls 112 and/or one or more reflector controls 114. The one or more controls 112, 114 can be pull wires. The one or more controls 112, 114 can be wireless controls. The transducer controls 112 can be independently and/or collectively articulated to control the position of the transducer (e.g., transducer 16). The reflector controls 114 can be independently and/or collectively articulated to control the position of the reflector 22 (e.g., first and second reflectors 22a, 22b). For example, FIG. 1D illustrates that the system 10 can have a transducer control 112 connected to the transducer 16, a first reflector control 114a connected to the first reflector 22a, and/or a second reflector control 114b connected to the second reflector 22b.

The controls 112, 114 can be used to translate and/or rotate the elements to which they are connected along and/or relative to the longitudinal axis 40 and/or to each other. The first reflector control 114a can be articulated (e.g., pulled, pushed, twisted) to translate the first reflector 22a toward and/or away from the second reflector 22b and/or the transducer 16. The second reflector control 114b can be articulated (e.g., pulled, pushed, twisted) to translate the second reflector 22b toward and/or away from the first reflector 22a and/or the transducer 16. For example, the first reflector control 114a can be pulled to translate the first reflector 22a away from (e.g., longitudinally away from) the second reflector 22b and/or the transducer 16. The first reflector control 114a can be pushed to translate the first reflector 22a toward (e.g., longitudinally toward) the second reflector 22b and/or the transducer 16. The second reflector control 114b can be pulled to translate the second reflector 22b toward (e.g., longitudinally toward) the first reflector 22a and/or the transducer 16. The second reflector control 114b can be pushed to translate the second reflector 22b away from (e.g., longitudinally away from) the first reflector 22a and/or the transducer 16. As described above, this adjustability can advantageously allow the ultrasound device 12 to deliver ultrasound energy to tissue at one or more treatment depths. For example, the treatment depth can be greater when the first and second reflectors 22a, 22b are farther apart than when closer together, relative to, for example, the longitudinal axis 40 of the device 12 and/or a blood vessel wall.

The first reflector control 114a can be articulated (e.g., pulled, pushed, twisted) to rotate the first reflector 22a relative to the second reflector 22b and/or the transducer 16. The second reflector control 114b can be articulated (e.g., pulled, pushed, twisted) to rotate the second reflector 22b relative to the first reflector 22a and/or the transducer 16.

FIG. 1D illustrates that the device 12 can have a third shaft 44c in addition to the first and second shafts 44a. 44b described above. FIG. 1D illustrates that the first, second, and third shafts 44a, 44b, 44c can have the arrangement shown. At least a portion of the second shaft 44b can be within a lumen of the first and third shafts 44a, 44c. At least a portion of the third shaft 44c can be within a lumen of the first shaft 44a. The third shaft 44c can be a polyimide isolator. The third shaft 44c can be moveable like the first and second shafts 44a, 44b described above, for example, with respect to the first and/or second shafts 44a, 44b.

FIG. 1E is a magnified view of the system of FIG. 1B at section 1E-1E.

FIG. 1F illustrates the ultrasound device 12 of FIG. 1C without the shield 32. FIG. 1F illustrates that the device 12 can have a distal diameter 68 and a proximal diameter 70. The distal diameter 68 can be from about 0.050 inches to about 0.150 inches (e.g., about 0.088 inches). The proximal diameter 70 can be from about 0.050 inches to about 0.150 inches (e.g., about 0.100 inches). The distal and proximal diameters 68, 70 can have the same or different dimensions. FIG. 1F illustrates that the distance between the first and second reflectors 22a, 22b can have a reflector-to-reflector separation 72 (also referred to as an offset gap). The separation 72 can be from about 0.020 inches to about 0.500 inches (e.g., about 0.050 inches, about 0.060 inches, about 0.070 inches, about 0.080 inches, about 0.090 inches, about 0.100 inches, about 0.110 inches, about 0.120 inches, about 0.130 inches, about 0.140 inches, about 0.150 inches, about 0.160 inches, about 0.170 inches, about 0.180 inches, about 0.190 inches, about 0.200 inches, about 0.210 inches, about 0.220 inches, about 0.230 inches, about 0.240 inches, about 0.250 inches, 0.260 inches, 0.270 inches, about 0.280 inches, about 0.290 inches). The first separation 72 can be fixed. The first separation 72 can be adjustable, e.g., increased and/or decreased. For example, the first separation 72 can be increased and/or decreased by translating the first and/or second reflectors 22a, 22b as described above (e.g., translating the first and/or second reflector surfaces 24a, 24b relative to one another). FIG. 1F illustrates that the device 12 can have a first reflector-to-transducer separation 74a between the first reflector 22a and the first emitter surface 18a. The separation 74a can be from about 0.025 inches to about 0.125 inches (e.g., about 0.084 inches). The separation 74a can be adjustable, e.g., increased and/or decreased. For example, the separation 74a can be increased and/or decreased by translating and/or rotating at least a portion of the first reflector 22a (e.g., reflective surface 24a) toward and/or away from the transducer 16 and/or vice versa as described above. FIG. 1F illustrates that the device 12 can have a second reflector-to-transducer separation 74b between the second reflector 22b and the second emitter surface 18b. The separation 74b can be from about 0.025 inches to about 0.250 inches (e.g., about 0.082 inches, about 0.125 inches). The separation 74b can be adjustable. e.g., increased and/or decreased. For example, the separation 74b can be increased and/or decreased by translating and/or rotating at least a portion of the second reflector 22b (e.g., reflective surface 24b) toward and/or away from the transducer 16 and/or vice versa as described above. The separations 74a, 74b can have the same or different dimensions relative to one another.

FIG. 1G illustrates the ultrasound device of FIG. 1D without the shield 32. FIG. 1G illustrates that the device 12 can have one or more wires 76. For example, the device 12 can have first and second wires 76a, 76b electrically connected to the transducer via, for example, the first and second bonds 60a, 60b, respectively. The first wire 76a can be electrically connected to, or otherwise be in electrical communication with, the first emitter surface 18a. The second wire 76b can be electrically connected to, or otherwise be in electrical communication with, the second emitter surface 18b. The one or more wires 76 can be in a lumen of the first, second, and/or third shafts 44a, 44b, 44c. For example, the first and/or second wires 76a, 76b can be in a space between the second and third shafts 44b, 44c, a space between the first and third shafts 44a, 44c and/or a space between the first and second shafts 44a, 44b. FIG. 1G illustrates that the first and second wires 76a, 76b can be between an inner surface of the third shaft 44c and an outer surface of the second shaft 44b. The one or more wires 76 can be in a shaft wall.

FIG. 1G illustrates that the first shaft 44a can have a first shaft length 44a_{L}. The first shaft length 44a_{L} can be from about 0.100 inches to about 0.200 inches (e.g., about 0.155 inches). FIG. 1G illustrates that the second shaft 44b can have a second shaft length 44b_{L}. The second shaft length 44b_{L} can be 0.100 inches to about 0.800 inches (e.g., about 0.460 inches). FIG. 1G illustrates that the third shaft 44c can have a third shaft length 44c_{L}. The third shaft length 44c_{L} can be from about 0.110 inches to about 0.250 inches (e.g., about 0.195 inches). FIG. 1G illustrates that the third shaft 44c can have a third shaft proximal length 82 proximal the transducer 16. The third shaft proximal length 82 proximal the transducer 16 can be from about 0.130 inches to about 0.230 inches (e.g., about 0.185 inches). FIG. 1G illustrates that a portion of the proximal end of the third shaft 44c can extend past an inside surface of the first reflector 22a by a third shaft dimension 84. The third shaft dimension 84 can be from about 0.005 inches to about 0.050 inches (e.g., 0.015 inches). FIG. 1G illustrates that a portion of the proximal end of the second shaft 44b can extend past an inside surface of the first reflector 22a by a second shaft dimension 86. The second shaft dimension 86 can be from about 0.005 inches to about 0.100 inches (e.g., 0.052 inches). FIG. 1G illustrates that a distal end of the second shaft 44c can be flush or nearly flush with the distal end (e.g., distal terminal end) of the second reflector 22b.

FIG. 1G illustrates that the transducer 16 can have a transducer outer diameter 16_{OD} and a transducer inner diameter 16_{ID}. The transducer outer diameter 16_{OD} can be from about 0.030 inches to about 0.170 inches (e.g., about 0.080 inches). The transducer inner diameter 16_{ID} can be from about 0.005 inches to about 0.160 inches (e.g., about 0.025 inches, about 0.080 inches, about 0.100 inches). The larger the emitter surface that the transducer 16 has, the more energy that the transducer 16 can emit. The first emitter surface 18a can have the same transducer outer diameter 16_{OD} as the second emitter surface 18b. The first emitter surface 18a can have a different transducer outer diameter 16_{OD} than the second emitter surface 18b, for example, smaller or larger. The first emitter surface 18a can have the same transducer inner diameter 16_{ID} as the second emitter surface 18b. The first emitter surface 18a can have a different transducer inner diameter 16_{ID} than the second emitter surface 18b, for example, smaller or larger.

FIG. 1G illustrates that the first reflector 22a can have a first reflector outer diameter 22a_{OD} and a first reflector inner diameter 22a_{ID}. The first reflector outer diameter 22a_{OD} can be from about 0.030 inches to about 0.180 inches (e.g., about 0.080 inches). The first reflector inner diameter 22a_{ID} can be from about 0.005 inches to about 0.170 inches (e.g., about 0.015 inches). FIG. 1G illustrates that the second reflector 22b can have a second reflector outer diameter 22b_{OD} and a second reflector inner diameter 22b_{ID}. The second reflector outer diameter 22b_{OD} can be from about 0.030 inches to about 0.180 inches (e.g., about 0.080 inches). The second reflector inner diameter 22b_{ID} can be from about 0.005 inches to about 0.170 inches (e.g., about 0.015 inches). The first reflector outer diameter 22a_{OD} can be the same or different from the second reflector outer diameter 22b_{OD}. The first reflector inner diameter 22a_{ID} can be the same or different from the second reflector inner diameter 22b_{ID}. The transducer outer diameter 16_{OD} can be the same or different from the first reflector outer diameter 22a_{OD}. The transducer outer diameter 16_{OD} can be the same or different from than the second reflector outer diameter 22b_{OD}. As shown, the transducer outer diameter 16_{OD} can be less than each of the first and second reflector out diameters 22a_{OD}, 22b_{OD}. This dimensional arrangement can help ensure that all of the ultrasound energy emitted toward the first and second reflectors 22a, 22b is redirected by the first and second reflectors 22a, 22b.

FIG. 1H illustrates a transparent view of the device 12 of FIG. 1C.

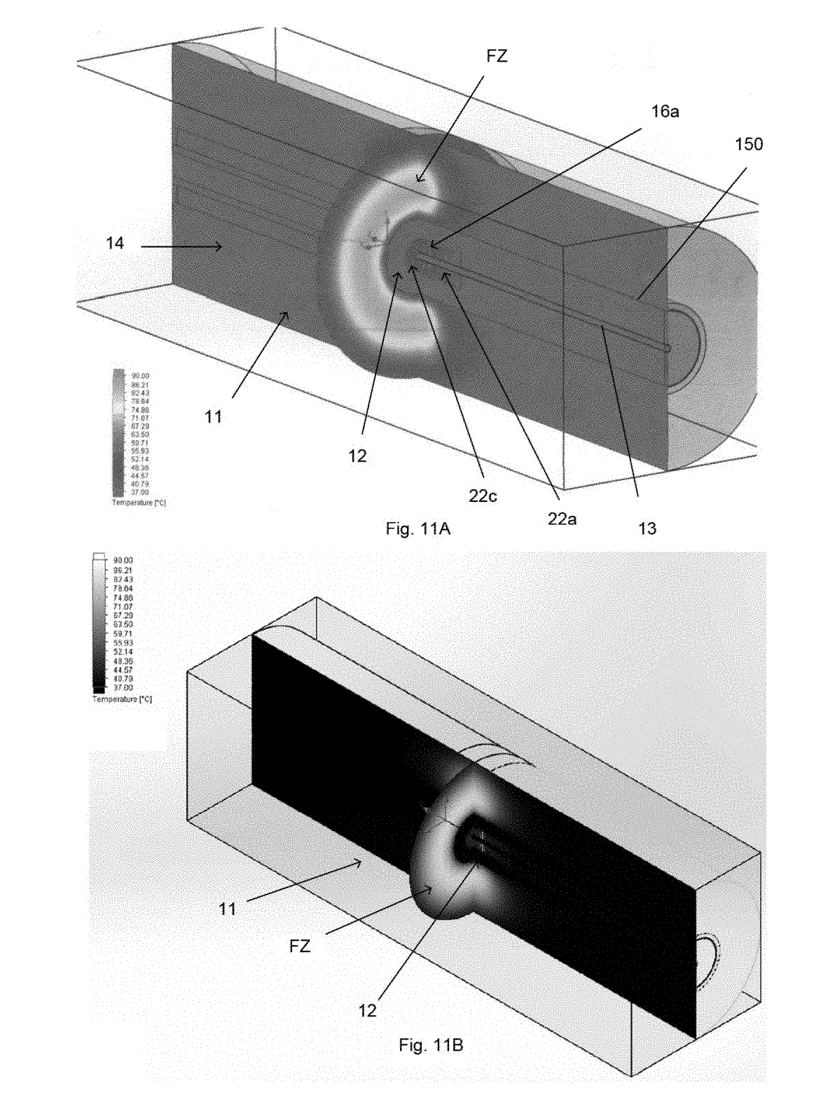

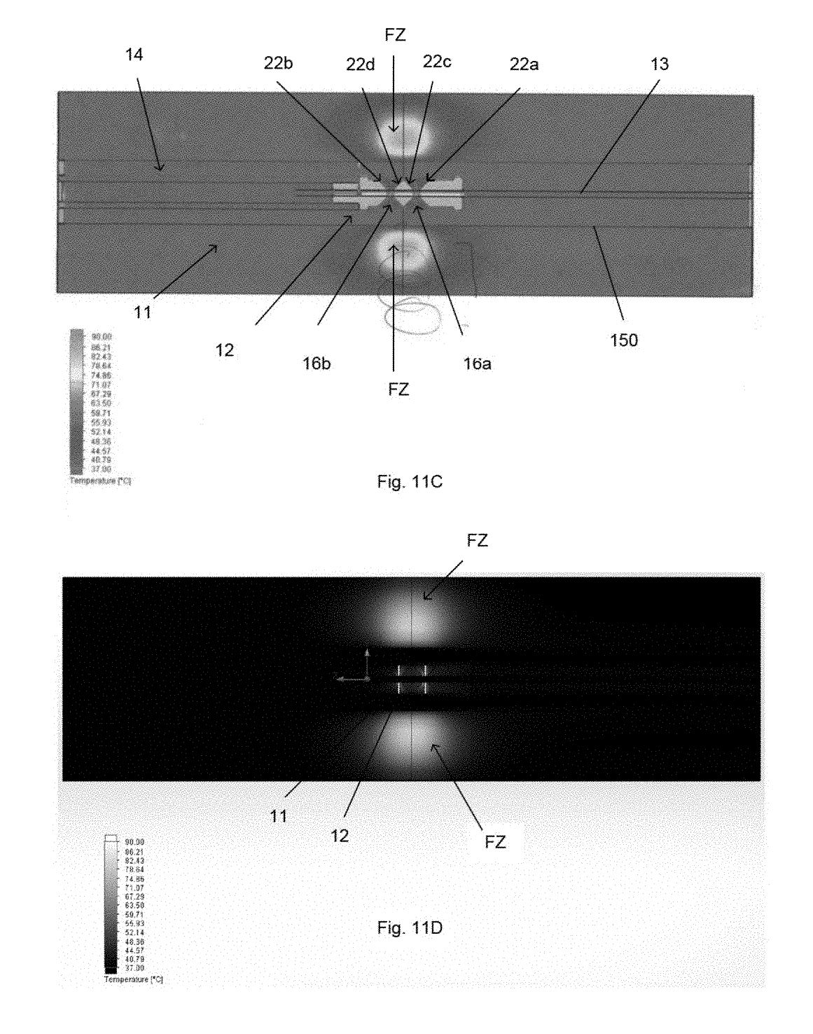

FIG. 1I illustrates that a shaft bond 37 can extend around the second shaft 44b. The shaft bond 37 can be an epoxy (e.g., conductive epoxy. The shaft bond 37 can seal around one or more wires 76 (e.g., first and/or second wires 76a, 76b). FIG. 11I illustrates that the device 12 can have a first shaft-first reflector bond 75a. The first shaft-first reflector bond 75a can attach the first shaft 44a to the first reflector 22a. The first shaft-first reflector bond 75a can be a glue or adhesive. FIG. 1I illustrates that the device 12 can have a second shaft-second reflector bond 75b. The second shaft-second reflector bond 75b can attach the second shaft 44b to the second reflector 22b. The second shaft-second reflector bond 75b can be a glue or adhesive. The first shaft 44 can be moved (e.g., telescoped) longitudinally along the second and/or third shafts 44b, 44c. The first and/or second reflectors 22a, 22b can be moved as described above. For example, the first and/or second reflectors 22a, 22b can be translated along the longitudinal axis. The first and/or second reflector surfaces 24a, 24b can be translated along the longitudinal axis.

FIG. 1J illustrates that the system 10 can have a guidewire 13. FIG. 1J illustrates that the device 12 can be delivered over the guidewire 13. The guidewire 13 can pass through the device 12. For example, the guidewire 13 can pass through the transducer 16, the first and second reflectors 22a, 22b, the first, second, and third shafts 44a, 44b, 44c, the coupler 48, and the catheter shaft 52.

FIG. 1K illustrates that the guidewire 13 can pass through the transducer 16, the second reflector 22b, and the first, second, and third shafts 44a, 44b, 44c.

FIG. 1L illustrates that the guidewire 13 can pass through the first reflector 22a and the coupler 48. FIG. 1L illustrates that the first shaft-first reflector bond 75a can have a first portion 75a₁ distal to a second portion 75a2. The first and second portions 75a₁, 75a₂ can be the same or a different type of bond. The first and second portions 75a₁, 75a₂ can be the same or different type of glue or adhesive. FIG. 1L illustrates that the device 12 can have a wire shield 78 adjacent or near the one or more wires 76.

FIG. 1M illustrates that the device 12 can be delivered over a guidewire 13.

FIG. 1N illustrates a variation of a transverse cross-sectional view of the device 12 of FIG. 1M taken along line 1N-1N with the features in the arrangement shown.

FIG. 1O illustrates a variation of a transverse cross-sectional view of the device 12 of FIG. 1M taken along line 1O-1O with the features in the arrangement shown.

FIG. 1P illustrates a variation of a transverse cross-sectional view of the device 12 of FIG. 1M taken along line 1P-1P with the features in the arrangement shown. FIG. 1P illustrates that the device 12 can have a solder connection 77. The solder connection 77 can attach a wire to a conductive tubing.

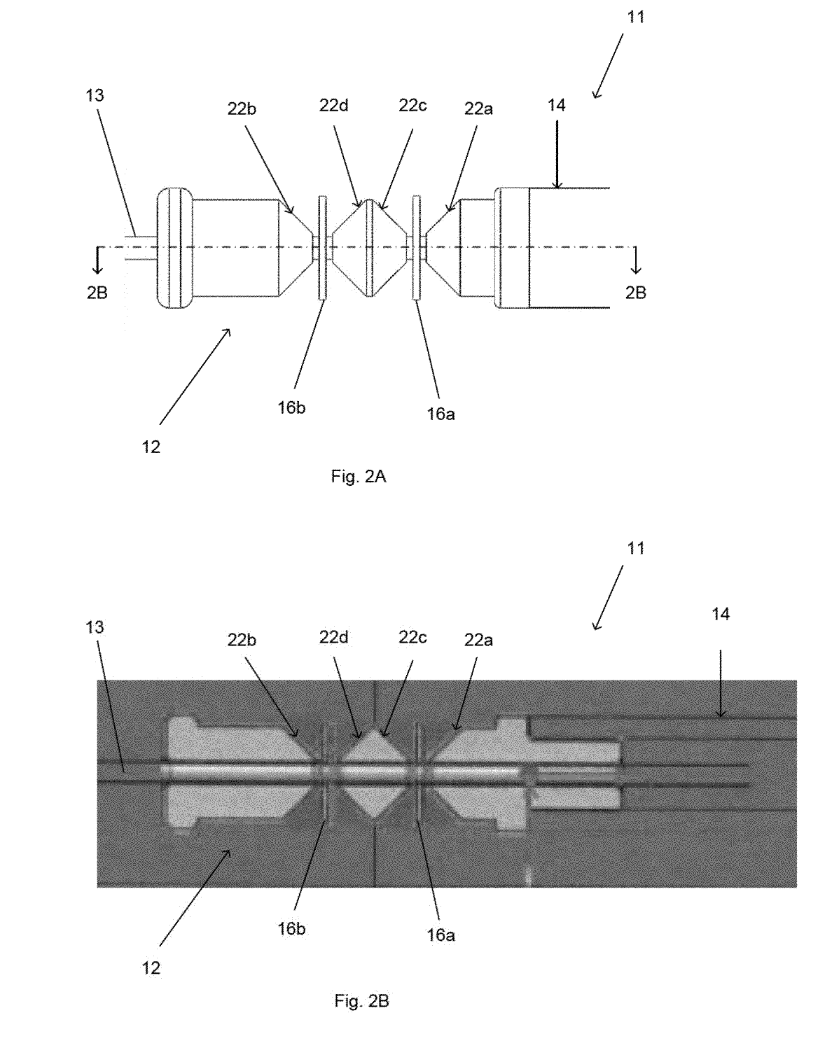

FIG. 2A illustrates that the device 12 can have two transducers and four reflectors, for example, a first transducer 16a, a second transducer 16b, a first reflector 22a, a second reflector 22b, a third reflector 22c, and a fourth reflector 22d. The first and second transducers 16a, 16b can be identical or functionally equivalent to the one or more transducers (e.g., transducer 16) described above. The third and fourth reflectors 22c, 22d can be identical or functionally equivalent to the one or more reflectors (e.g., first and second reflectors 22a, 22b) described above. FIG. 2A illustrates that the device 12 can be delivered over a guidewire 13. The device 12 can have a shield 32 (not shown).

FIG. 2B illustrates that the guidewire 13 can pass through a longitudinal center of the device 12.

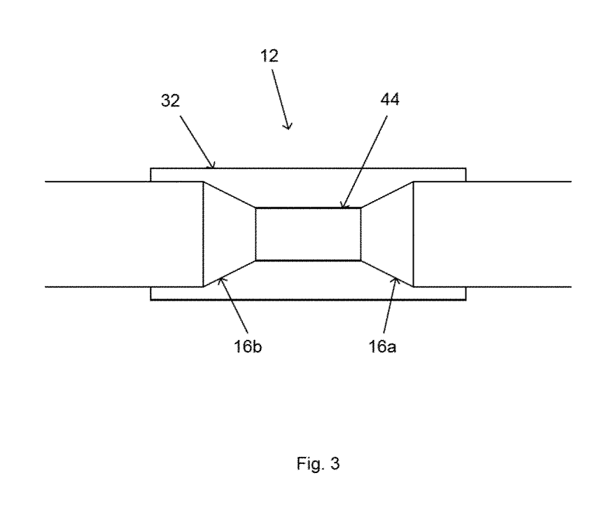

FIG. 3 illustrates that the ultrasound device 12 can have one or multiple transducers that can have a tapered three-dimensional shape similar to that described above with respect to first and second reflectors 22a, 22b. For example, as shown, the device 12 can have a first transducer 16a and a second transducer 16b. The first and second transducers 16a, 16b can have a conical, truncated-conical, or a frusto-conical shape. For example, as shown, the first and second transducers 16a, 16b can have a frusto-conical shape. The first and second transducers 16a, 16b can have the same or different shape relative to one another. Although not shown in FIG. 3, one or more reflectors can be used with the variation shown in FIG. 3 to target an ablation site. The first and second transducers 16a, 16b can be translated and/or rotated relative to one another and the longitudinal axis 40.

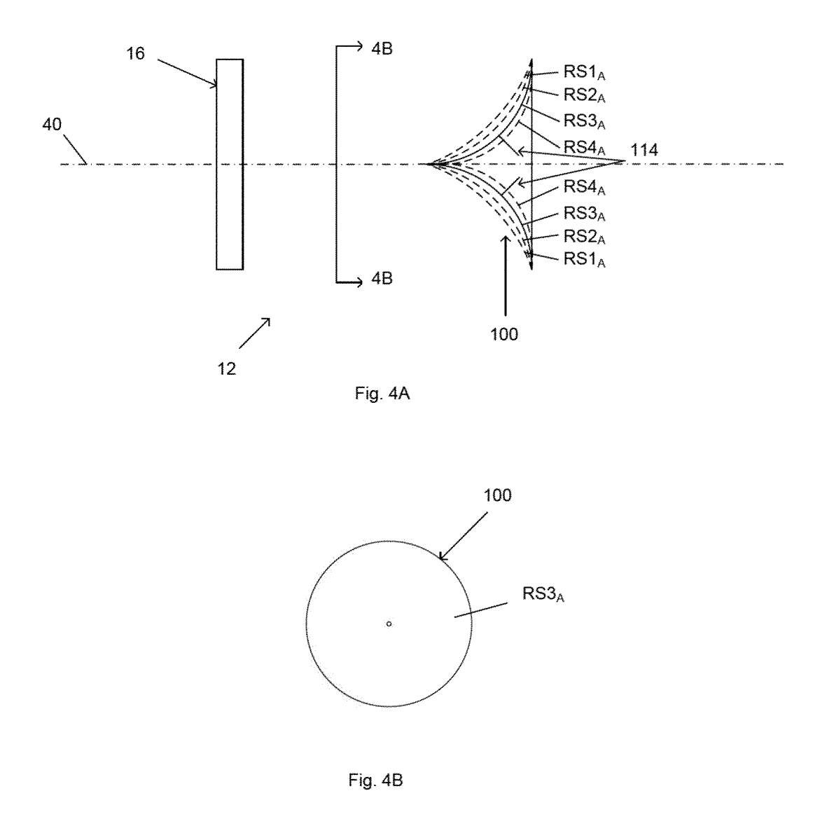

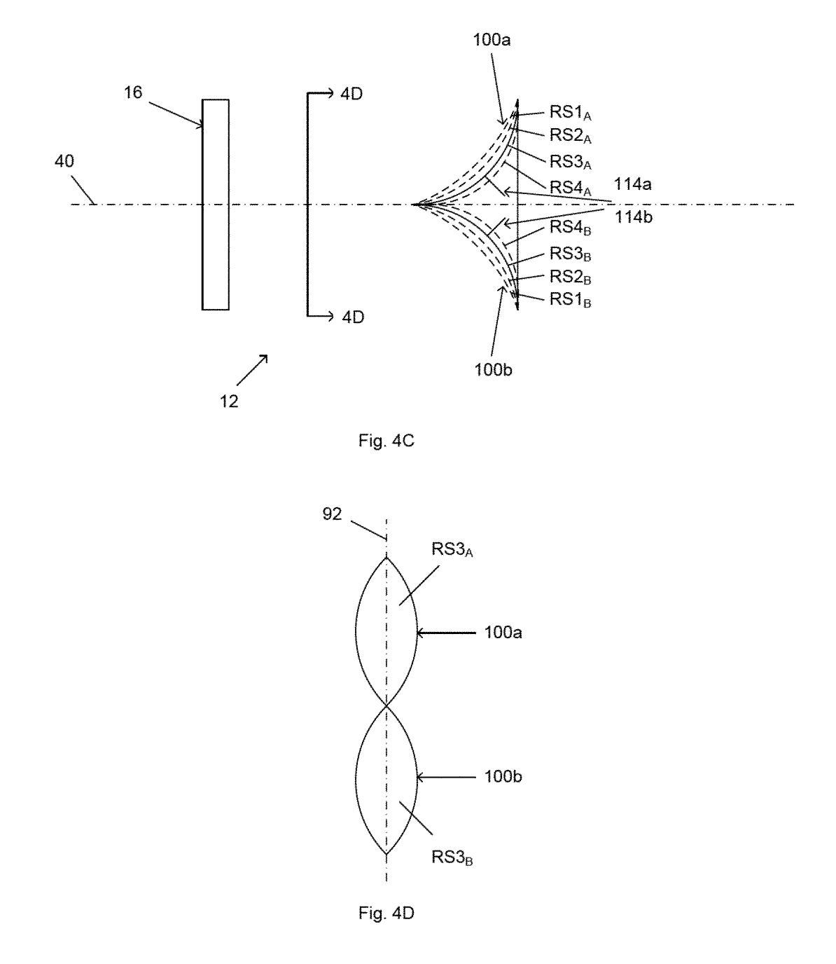

FIG. 4A illustrates that the system 10 can have a transducer 16 and one or more adjustable reflectors 100. The reflective surface of the adjustable reflector 100 can be adjusted into different surface geometries, including one or more concave, flat, and/or convex shapes. At least a portion of the adjustable reflector 100 can be concave, flat, and/or convex in each of the different surface geometries. For example, FIG. 4A illustrates that the adjustable reflector 100 can have a convex reflective surface RS3_{A} that can be adjusted to any of the convex surfaces RS1_{A}-RS4_{A}, including any position between or beyond these illustrated surfaces, for example, into one or more flat and/or convex surface geometries (not shown but easily appreciated). As shown, each of the reflective surfaces RS1_{A}-RS4_{A} can have a different radius of curvature. The adjustable reflector 100 can have, for example, a conical, truncated-conical, or a frusto-conical shape with the adjustable reflective surface described above; however, other shapes are also appreciated (see e.g., FIG. 4D). One or multiple reflector controls 114 can be directly or indirectly connected to the adjustable reflector 100 to move the reflective surface RS into different surface geometries, e.g., RS1_{A}-RS4_{A}.

FIG. 4B illustrates that the adjustable reflector 100 can extend at least partially around the longitudinal axis 40.

FIG. 4C illustrates that the device 12 can have multiple adjustable reflectors 100. For example, FIG. 4C illustrates that the device 12 can have first adjustable reflector 100a and a second adjustable reflector 100b. FIG. 4D is a variation of a top view of the multiple adjustable reflectors 100a, 100b of FIG. 4C taken along line 4D-4D. FIGS. 4C and 4D are similar to FIGS. 4A and 4B except two adjustable reflectors 100a, 100b are shown. The first and second adjustable reflectors 100a, 100b in FIGS. 4C and 4D can be adjusted to any of the convex surfaces RS1_{A}-RS4_{A}, RS1_{B}-RS4_{B}, respectively, including any position between or beyond these illustrated surfaces, for example, into one or more flat and/or convex surface geometries (not shown but easily appreciated). FIG. 4D illustrates that the first and second adjustable reflectors 100a, 100b can be positioned opposite one another along a transverse axis 92 that intersects the longitudinal axis 40.

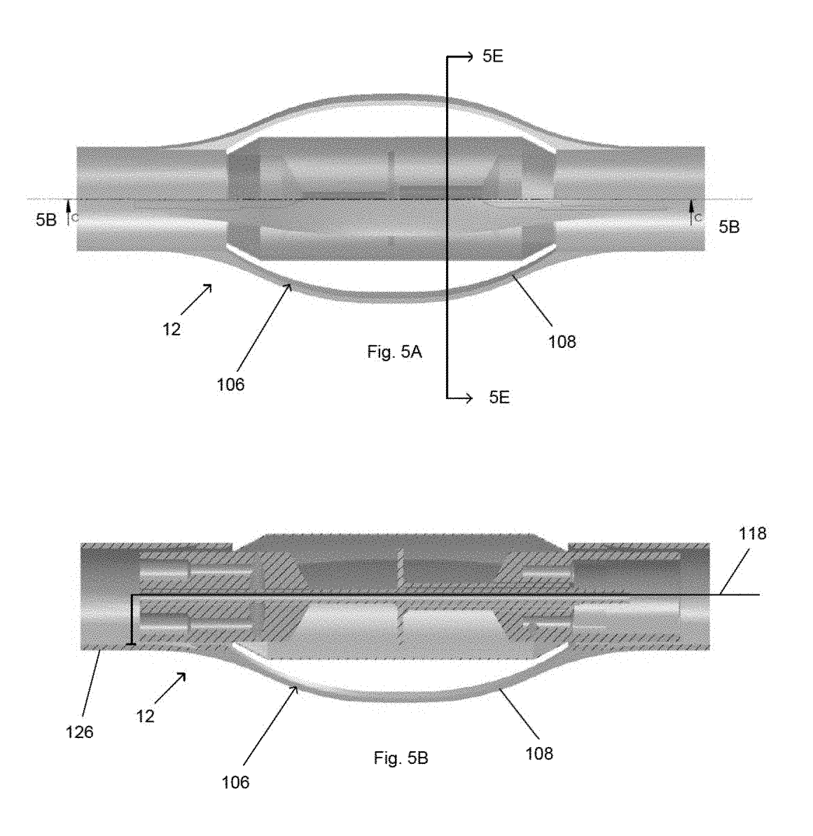

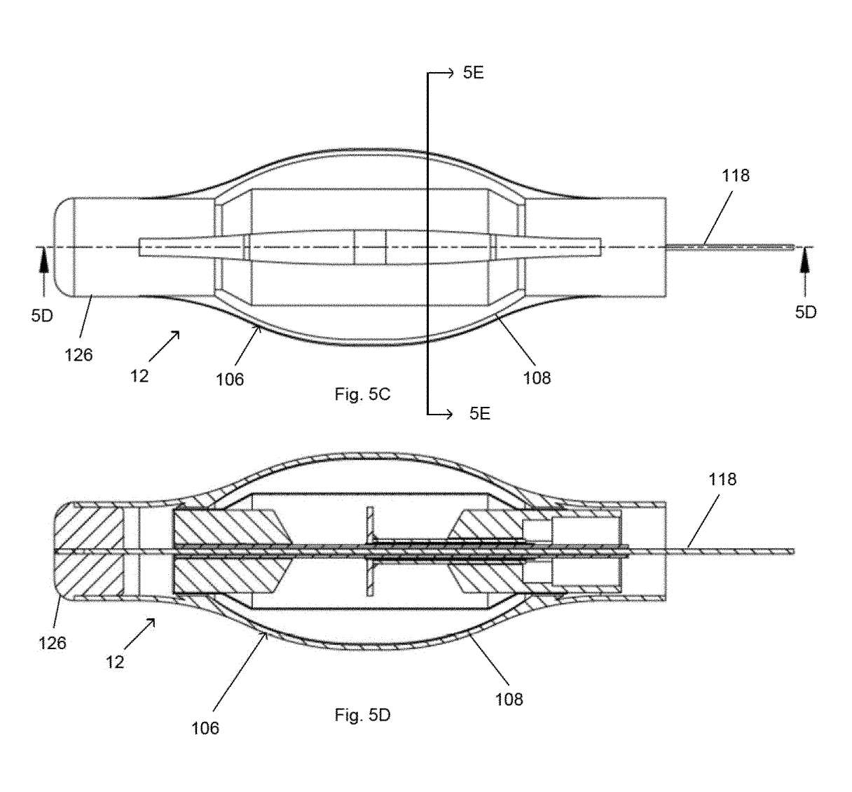

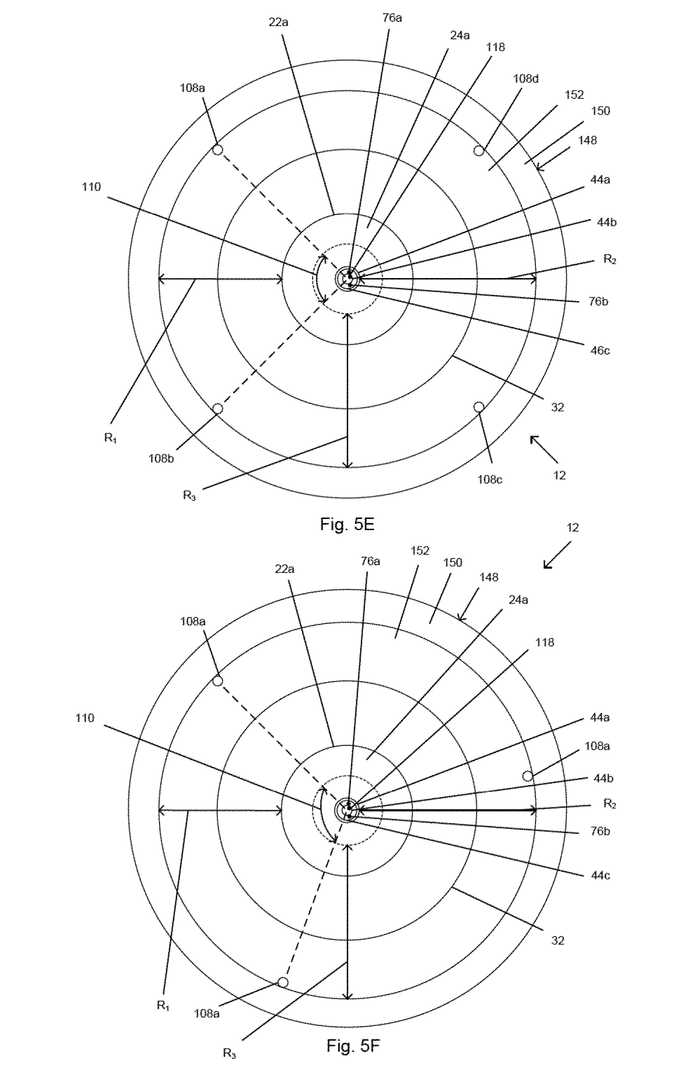

FIGS. 5A-5F that the ultrasound device 12 can have a spacer 106. The spacer 106 can be attached to the device 12. The spacer 106 can have one or more splines 108, for example, 1 to 10 or more. The spacer can be transparent to ultrasound energy. For example, the splines 108 can be transparent to ultrasound energy.

The spacer 106 can be collapsible and/or expandable. The splines 108 can be expanded to abut the internal wall of a blood vessel. The one or more splines 108 can position the ultrasound device 12 in a lumen of a blood vessel so that the nerves to be ablated are properly targeted. The one or more splines 108 can position the ultrasound device 12 one or more radial distances from a blood vessel wall. The spacer 106 can be adjusted to match the internal radii or diameter(s) of a blood vessel. For example, the spacer 106 can be adjusted to have one or multiple radii or diameters. The one or more splines 108 can have a radius from the center of the device 12 from about 1.5 mm to about 4.5 mm (e.g., about 2.2 mm), for example, as measured from the middle of each of the splines 108, from the surface of the splines contacting a vessel wall, or from the surface of the splines facing toward the center of the device 12. The one or more splines 108 can have a diameter from the center of the device 12 from about 3 mm to about 9 mm (e.g., about 4.3 mm), for example, as measured from the middle of each of the splines 108, from the surface of the splines contacting a vessel wall, or from the surface of the splines facing toward the center of the device 12. The spacer 106 can be adjusted to stretch the internal diameter(s) of a blood vessel. For example, the spacer 106 can be adjusted to have one or multiple diameters from about 3 mm to about 9 mm (e.g., about 7.8 mm). FIGS. 5A-5D illustrate that the splines 108 can form arcs with a flat or pointed apexes. This ability of the spacer 106 to have multiple radii and/or diameters advantageously allows the spacer 106 to accommodate vessels that do not have cylindrical lumens, for example, due to plaque buildup.

FIG. 5B illustrates that the one or more splines 108 can be independently or collectively (e.g., two or more simultaneously) articulated with a spacer control 118 to expand and/or collapse the spacer 106 to a desired diameter. FIG. 5B illustrates that the distal ends of the splines 108 can be attached to the second longitudinal end 38 of the device 12 and that the proximal ends of the splines can be attached to the first longitudinal end 36 of the device 12. The distal ends of the splines 108 can be integrated with the second longitudinal end 38 of the device 12 (e.g., a portion of the second reflector 22b). The proximal ends of the splines 108 can be integrated with the first longitudinal end 36 of the device 12 (e.g., a portion of the first reflector 22a).

FIG. 5B illustrates that the spacer 106 can have a cap 126 with an open end. The cap 126 can be attached to or integrated with the second longitudinal end 38 of the device 12. The distal ends of the splines 108 can be attached to or integrated with the cap 126.

The spacer control 118 can translate the second longitudinal end 38 of the device 12 relative to the first longitudinal end 36, vice versa, and/or can translate both the first and second longitudinal ends 36, 38. The spacer 106 can collapse when the distance between first and second longitudinal ends 36, 38 is increased. For example, the spacer 106 can collapse when the second longitudinal end 38 is translated away from the first longitudinal end 36. The spacer 106 can expand when the distance between first and second longitudinal ends 36, 38 is decreased. For example, the spacer 106 can expand when the second longitudinal end 38 is translated toward the first longitudinal end 36.

The spacer control 118 can rotate the second longitudinal end 38 of the device 12 relative to the first longitudinal end 36, vice versa, and/or can rotate both the first and second longitudinal ends 36, 38. The first and/or second longitudinal ends 36, 38 can be rotated about, example, the longitudinal axis 40. The spacer 106 can collapse when the first and/or second longitudinal ends 36, 38 are rotated relative to one another such that the distance between the distal and proximal ends of the splines 108 is increased. For example, the spacer 106 can collapse when the second longitudinal end 38 is rotated in a first direction (e.g., clockwise) relative to the first longitudinal end 36. The spacer 106 can expand when the first and/or second longitudinal ends 36, 38 are rotated relative to one another such that the distance between the distal and proximal ends of the splines 108 is decreased. For example, the spacer 106 can expand when the second longitudinal end 38 is rotated in a second direction (e.g., counterclockwise) relative to the first longitudinal end 36.

The spacer control 118 can translate and/or rotate the second longitudinal end 38 of the device 12 relative to the first longitudinal end 36, vice versa, and/or can translate and/or rotate both the first and second longitudinal ends 36, 38.

The spacer 106 can be a basket. The spacer 106 can be an inflatable balloon. The splines 108 can be an articulable material and/or can be inflatable. The spacer 106 can be a wire mesh. A wire mesh can be positioned around the spacer 106, for example, which can be configured to advantageously capture potential embolic material dislodged by the spacer 106.

FIGS. 5C and 5D illustrate that the spacer 106 can have a cap 126 with a closed end. The cap 126 can be translated and/or rotated with the spacer control 118.

FIG. 5E illustrates that the device 12 can be in a blood vessel 148 having a lumen 152 and a blood vessel wall 150. FIG. 5E illustrates that the spacer 106 can have four splines 108a, 108b, 108c, 108d separated from one another by a spline angle 110. The spline angle 110 can be from about 5 degrees to 180 degrees, for example, about 30 degrees, about degrees, about 60 degrees, about 90 degrees, about 120 degrees, and/or about 180 degrees. The spline angle 110 can be the same or different between different adjacent splines 108. For example, FIG. 5E illustrates that the spline angle 110 can be about 90 degrees between each of the adjacent splines 108. FIG. 5E illustrates that the splines 108 can contact the inner surface of the blood vessel 148. As described above, the four splines 108a, 108b, 108c, 108d can have a radius from the center of the device 12 from about 1.5 mm to about 3.5 mm (e.g., about 2.2 mm), for example, as measured from the middle of each of the splines 108, from the surface contacting a vessel wall, or from the surface facing toward the center of the device 12. FIG. 5E illustrates that a first radius R₁ from the reflector outer diameter (e.g., 22a_{OD} and/or 22b_{OD}) to the vessel wall 150 can be 12 from about 0.015 inches to about 0.180 inches (e.g., about 0.040 inches). FIG. 5E illustrates that a second radius R₂ from the reflector inner diameter (e.g., 22a_{ID} and/or 22b_{ID}) to the vessel wall 150 can be from about 0.050 inches to about 0.180 inches (e.g., about 0.100 inches). FIG. 5E illustrates that a third radius R₃ from the radial middle of the reflector (represented by the dashed circle in FIG. 5E) to the vessel wall 150 can be from about 0.025 inches to about 0.160 inches (e.g., about 0.060 inches).

FIG. 5F illustrates that the spacer 106 can have three splines 108a, 108b, 108c. FIG. 5F illustrates that the spline angle 110 can be about 120 degrees between each of the adjacent splines 108a, 108b, 108c.

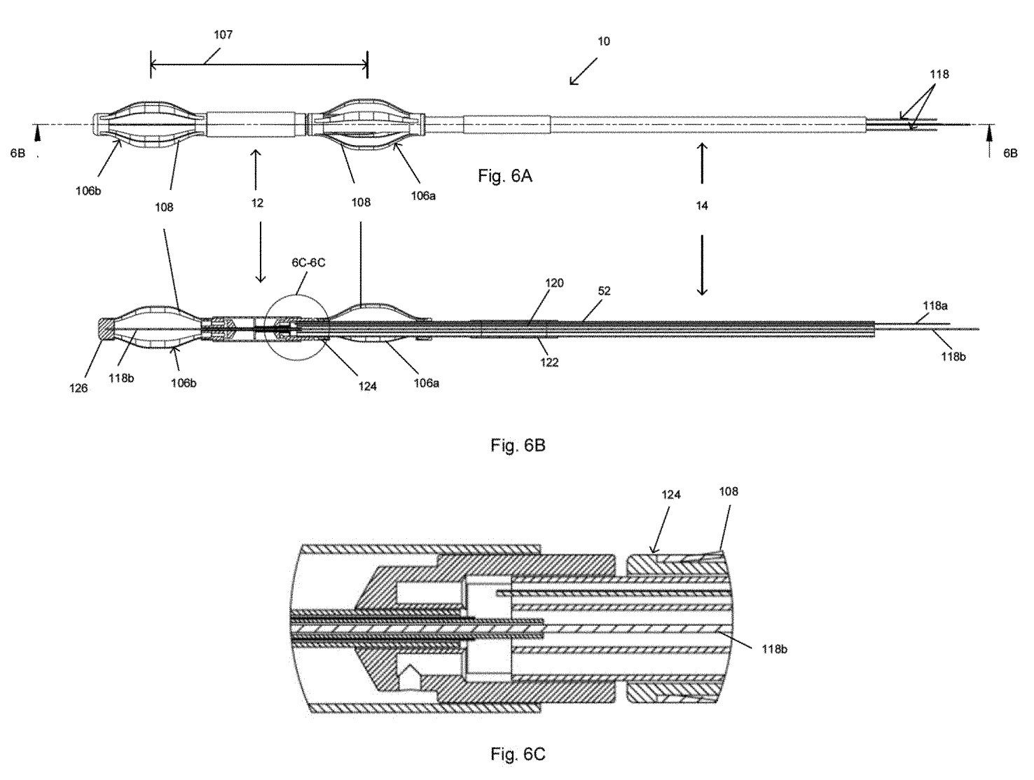

FIG. 6A illustrates that the system 10 can have a first spacer 106a and a second spacer 106b with the device 12 positioned therebetween. As shown, the first spacer 106a can be positioned distal to the device 12 and the second spacer 106b can be positioned proximal to the device 12. The first and second spacers 106a, 106b can be identical or functionally similar to the spacers (e.g., spacer 106) described above. The first and second spacers 106a and 106b can be the same or different from one another. For example, the first spacer 106a can have splines 108 and the second spacer 106b, although not shown, can be an inflatable balloon. FIG. 6A illustrates that the centers of the first and second spacers 106a, 106b can be separated by a separation dimension 107. The separation dimension 107 can be from about 0.500 inches to about 1.500 inches (e.g., about 1.000 inches). The spacers 106a, 106b can be controlled with spacer controls 118.

FIG. 6B illustrates that the first spacer 106a can have a sleeve 124 and that the second spacer 106b can have a cap 126. The first and second spacers 106a, 106b can be expanded and/or collapsed as described above with reference to spacer 106. For example, the sleeve and cap 124, 126 can be translated and/or rotated via first and second spacer controls 118a, 118b, respectively. The first and second spacers 106a, 106b can have the same or different diameters. FIG. 6B illustrates that the catheter 14 can have a catheter shaft segment 120 and a seal 122.

FIG. 6C is a magnified view of the system 10 of FIG. 6B at section 6C-6C. FIG. 6C illustrates a close-up view of the sleeve 124.

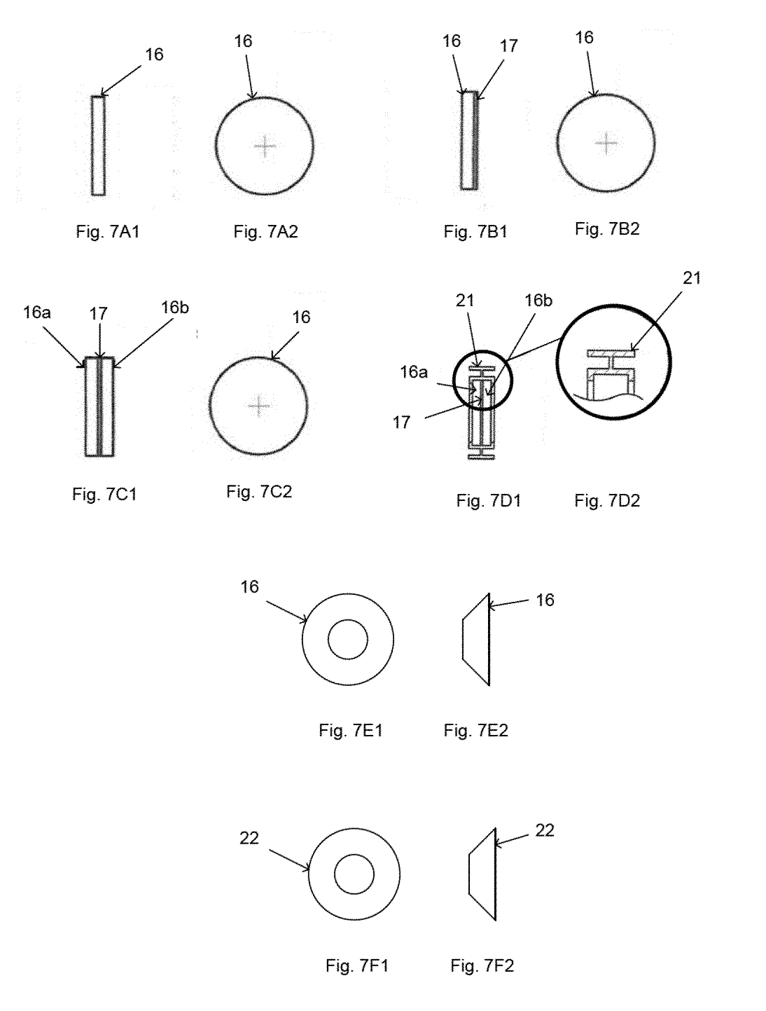

FIGS. 7A1 and 7A2 illustrate that the transducer 16 can be a one or two-sided transducer. The transducer 16 can have the shape of a disc, for example, an annular disc.

FIGS. 7B1 and 7B2 illustrate that the transducer 16 can have a backing layer 17. The backing layer 17 can be an insulating layer, for example, air and/or a micro balloon.

FIGS. 7C1 and 7C2 illustrate that a backing layer 17 can be between the first and second transducers 16a, 16b.

FIGS. 7D1 and 7D2 illustrate that the transducer 16 (e.g., first and second transducers 16a, 16b) can have a grommet 21. The grommet 21 can be an elastomer, for example, a thermoplastic elastomer.

FIGS. 7E1 and 7E2 illustrate that the transducer(s) 16 can have a frusto-conical shape, for example, as described above with reference to first and second transducers 16a, 16b shown in FIG. 3.

FIGS. 7F1 and 7F2 illustrate that the reflectors 22 (e.g., first and second reflectors 22a, 22b) can have a frusto-conical shape.

The device 12 can have an extra-small, small, medium, large, or extra-large size, advantageously allowing the device 12 to fit in the lumen of any sized blood vessel. For example, the devices 12 shown in the exemplary drawings can have the dimensions described above and below. The device 12 can be sized to fit in various vessel physiologies and apply therapeutic ablation treatment, for example, with the adjustability described above and below.

The systems (e.g., systems 10, 11) can perform diagnosis, imaging, and tissue structure sensing with one or more sensors, for example, one or more pressure sensors, temperature sensors, optical sensors, or combinations thereof, and/or with expandable/collapsible structures (e.g., balloon(s), stent(s)) having one or more of the foregoing sensors. For example, the device 12 can ablate tissue (e.g., by delivering ultrasound energy radially outward from the device) and/or sense one or more parameters. For example, the device 12 can sense tissue temperature (e.g., to determine/detect the amount of tissue burn) and/or treatment time (e.g., to determine/detect the amount of tissue burn). The device 12 can evaluate (e.g., sense) tissue condition ultrasonically, optically, and/or mechanically. The device 12 can alternate between treatment and sensing modalities and/or perform them simultaneously.

### Methods of Use

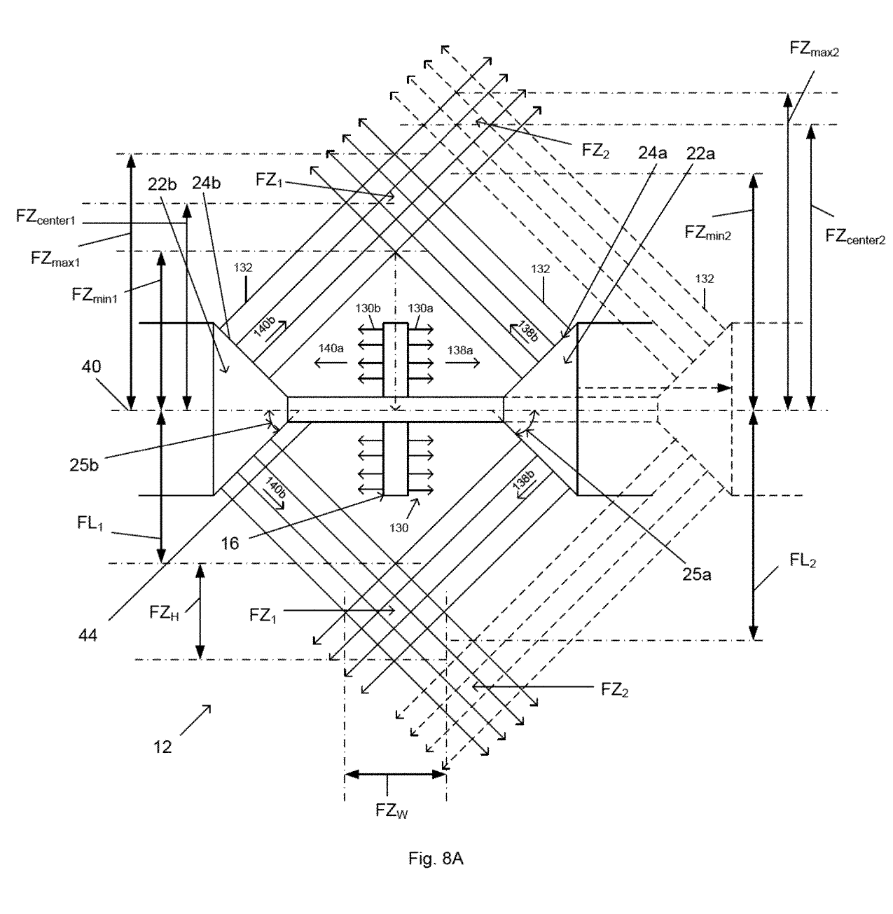

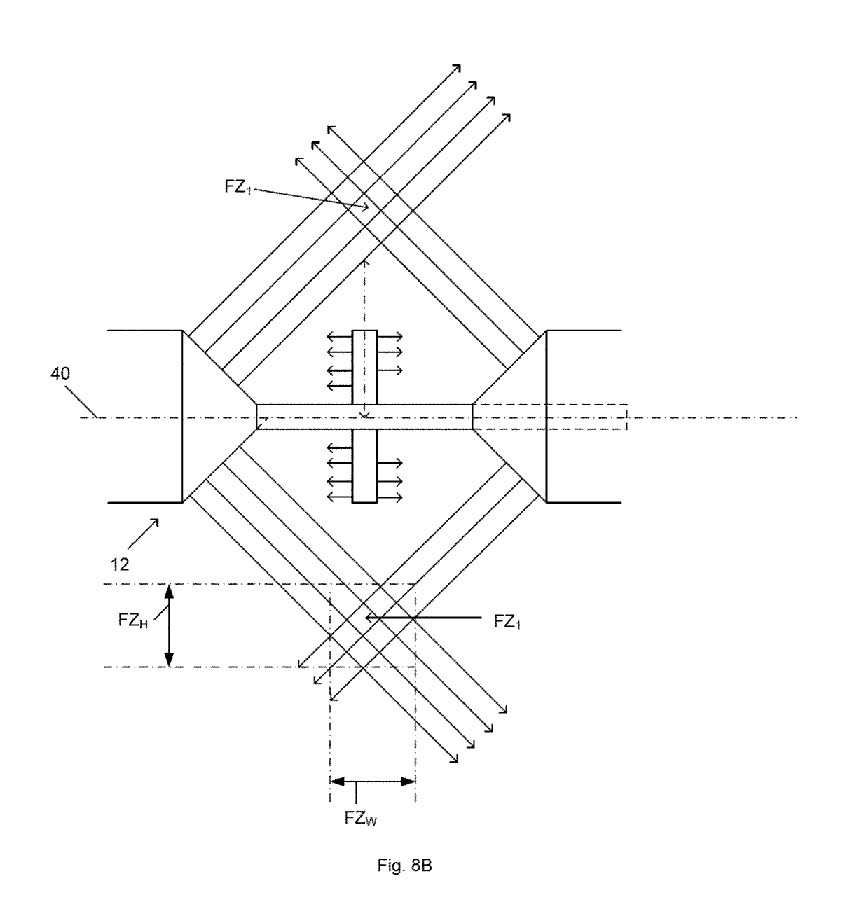

FIG. 8A illustrates that the ultrasound device 12 can emit ultrasound energy 130 via one or more transducers 16. The ultrasound device 12 can redirect emitted ultrasound energy 130 toward a focal zone FZ via one or more reflectors 22. The redirected ultrasound energy is labeled with numeral 132. As shown, the transducer 16 can emit ultrasound energy 130 in one or more beams (e.g., collimated beams) in one or more directions. For example, FIG. 8A illustrates that the transducer 16 can emit first and second ultrasound energy beams 130a, 130b. The first ultrasound energy beam 130a can be emitted in a first beam first direction 138a. The second ultrasound energy beam 130b can be emitted in a second beam first direction 140a. The first and second beam first directions 138a, 140a can be opposite directions from one another, or any non-parallel direction toward or away from each other. The first beam first direction 138a can be at a first beam first angle relative to the longitudinal axis 40, for example, from about 0 degrees to about 90 degrees (e.g., about 0 degrees). The second beam first direction 140a can be at a second beam first angle relative to the longitudinal axis 40, for example, from about 0 degrees to about 90 degrees (e.g., about 0 degrees). The first and second beam first angles can be the same or different from one another. The first and second beams 130a, 130b can extend at least partially around the longitudinal axis 40.

FIG. 8A illustrates that the first ultrasound energy beam 130a can be redirected, via the first reflector 22a, from the first beam first direction 138a to a first beam second direction 138b. As described above, the first reflective surface 24a can be at a first reflector surface angle 25a, which can be from about 30 degrees to about 60 degrees relative to the longitudinal axis 40, for example, about 45 degrees. The first beam second direction 138b can extend at least partially in a radial direction away from the longitudinal axis at a first beam second angle relative to the longitudinal axis 40. The first beam second direction 138b can be at a first beam second angle relative to the longitudinal axis 40, for example, from about 0 degrees to about 90 degrees (e.g., about 45 degrees). FIG. 8A illustrates that the second ultrasound energy beam 130b can be redirected, via the second reflector 22b, from the second beam first direction 140a to a second beam second direction 140b. As described above, the second reflective surface 24b can be at a second reflector surface angle 25b, which can be from about 30 degrees to about 60 degrees relative to the longitudinal axis 40, for example, about 45 degrees. The second beam second direction 140b can extend at least partially in a radial direction away from the longitudinal axis at a first beam second angle relative to the longitudinal axis 40. The second beam second direction 140b can be at a second beam second angle relative to the longitudinal axis 40, for example, from about 0 degrees to about 90 degrees (e.g., about 45 degrees).

FIG. 8A illustrates that the first beam 130a can be redirected at least partially in a radial direction away from the first reflector 22a. FIG. 8A illustrates that the second beam 130b can be redirected at least partially in a radial direction away from the second reflector 22b. FIG. 8A illustrates that the first beam 130a can be redirected at least partially in a longitudinal direction away from the first reflector 22a. FIG. 8A illustrates that the second beam 130b can be redirected at least partially in a longitudinal direction away from the second reflector 22b.

FIG. 8A illustrates that the first and second beams 130a, 130b can converge (also referred to as intersect) one or multiple radial distances away from the device 12 in a focal zone FZ. The first and second energy beams 130a, 130b can converge at the one or multiple focal zones FZ. The one or more focal zones can each extend at least partially around the longitudinal axis 40 of the device 12. The one or more focal zones FZ can each extend partially or completely around the longitudinal axis 40. The one or more focal zones FZ can each have a ring shape. The one or more focal zones FZ can each have a toroid shape (or similar shape). For example, FIG. 8A illustrates that the first beam 130a can extend toward a first focal zone FZ₁ in the first beam second direction 138b and the second beam 130b can extend toward the first focal zone FZ₁ in the second beam second direction 140b. At least a portion of the first and second beams 130a, 130b can intersect in the one or more focal zones FZ (e.g., the first focal zone FZ₁).

The radial center of the one or more focal zones FZ can coincide or be offset from the center of the device 12 (e.g., the longitudinal axis 40). For example, FIG. 8A illustrates that the radial center of the first and second focal zones FZ₁ and FZ₂ can coincide with the longitudinal axis 40 of the device.

FIG. 8A illustrates that the FZ can have a diamond-shaped cross-section, although any cross-section shape is appreciated, including circular, elliptical, ovoid, and/or polygonal. The FZ can have one or multiple cross-section shapes, for example, a circular-shape in a toroid first section and a diamond-shape in a toroid second section. The cross-sectional shape of the FZ can be changed from one shape to another (e.g., from diamond- to circular-shaped, from a first diamond shape to another diamond shape). FIG. 8A illustrates that the FZ can have a focal zone center FZ_{center}, a focal zone maximum FZₘₐₓ, a focal zone minimum FZₘᵢₙ, a focal zone width FZ_{W} and a focal zone height FZ_{H}. The FZ_{center} can be from about 3 mm to about 9 mm (e.g., about 7.2 mm) from the longitudinal axis 40. The FZₘₐₓ can be from about mm to about 9 mm (e.g., about 6.7 mm) from the longitudinal axis 40. The FZₘᵢₙ can be from about 3 mm to about 9 mm (e.g., about 7.7 mm) from the longitudinal axis 40. The FZ_{W} can be from about 0.3 mm to about 5 mm (e.g., about 0.5 mm) from the longitudinal axis 40. The FZ_{H} can be from about 0.3 mm to about 5 mm (e.g., about 0.5 mm) from the longitudinal axis 40. The FZ_{W} and the FZ_{H} can be the same or different from each other. The FZ can be partially or entirely outside a vessel wall.

As described above, two beams 130a, 130b of ultrasound energy can converge a radial distance away from the device 12. FIG. 8A illustrates that the first and second beams 130a. 130b can be redirected toward one or more focal zones FZ. The position of the focal zones FZ relative to the device 12 can be fixed or adjustable. The depth of the FZ in tissue can be adjustable. FIG. 8A illustrates that the FZ can be adjusted longitudinally and/or radially from a first focal zone FZ₁ to a second focal zone FZ₂. As shown, the first focal zone FZ₁ can have a first focal zone center FZ_{center1}, a first focal zone max FZₘₐₓ₁ and a first focal zone minimum FZₘᵢₙ₁. The second focal zone FZ₂ can have a second focal zone center FZ_{center2}, a second focal zone max FZₘₐₓ₂ and a second focal zone minimum FZₘᵢₙ₂. The various dimensions of the first and second focal zones FZ₁, FZ₂ can be greater than, less than and/or equal each other. For example, FIG. 8A illustrates that FZ_{center2} can be greater than FZ_{center1}, that FZₘₐₓ₂ can be greater than FZₘₐₓ₁ and that that FZₘᵢₙ₂ can be greater than FZₘᵢₙ₁.

FIG. 8A illustrates that the FZ can be moved from a first focal length FL₁ from the longitudinal axis 40 to a second focal length FL₂ from the longitudinal axis. The first focal length FL₁ can correspond to any dimension of the first focal zone FZ₁ (e.g., FZ_{center1}, FZₘₐₓ₁ and/or FZₘᵢₙ₁). As shown, the first focal length FL₁ can correspond to FZₘᵢₙ₁. The second focal length FL₂ can correspond to any dimension of the second focal zone FZ₂ (e.g., FZ_{center2}, FZₘₐₓ₂ and/or FZₘᵢₙ₂). As shown, the second focal length FL₂ can correspond to FZₘᵢₙ₂.

FIG. 8A illustrates that the focal zones (e.g., FZ₁ and FZ₂) can be symmetrical about two axes of symmetry when viewed in cross-section.

The foregoing focal zone adjustability can be accomplished by translating and/or rotating one or more transducers and one or more reflectors relative to one another and/or relative to the longitudinal axis 40. For example, the transducer 16, the first reflector 22a, and/or the second reflector 22b can be individually and/or collectively translated and/or rotated relative to each other and/or relative to the longitudinal axis 40. At least a portion of the transducer 16 can be translated and/or rotated toward and/or away from the first reflector 22a and/or vice versa. At least a portion of the transducer 16 can be translated and/or rotated toward and/or away from the first reflector 22b and/or vice versa. At least a portion of the first reflector 22a can be translated and/or rotated toward and/or away from the second reflector 22b and/or vice versa. At least a portion of the first reflector 22a can be translated and/or rotated toward and/or away from the transducer and/or vice versa. For example, FIG. 8A illustrates that the first reflector 22a is translated away from the transducer 16 and the second reflector 22b, thereby moving the FZ from FZ₁ to FZ₂. The device 12 can have one or multiple configurations. FIG. 8A illustrates exemplary first and second configurations of the device. As shown, the first reflector 22a is closer to the transducer 16 and the second reflector 22b along the longitudinal axis 40 in the first configuration than in the second configuration. In this way, ultrasonic energy emitted from transducer(s) 16 can be redirected by the one or more reflectors 22 (e.g., first and second reflectors 22a, 22b) to intersect in one or more adjustable focal zones FZ (e.g., first and second focal zones FZ₁, FZ₂).

The first and second beams 130a, 130b can have the same or different energy levels. For example, as described above, the first and second beams 130a, 130b can each have a harmonic frequency from about 9 MHz to about 15 MHz.

The ultrasound energy density (also referred to as energy level) in the FZ can be higher than the ultrasound energy density outside of the FZ. Within the FZ, the energy density can be the greatest in the center of the FZ and can be the least along the periphery of the FZ. However, it will be appreciated that any suitable energy density distribution is appreciated, for example, high energy density on the half of the FZ farther from the device 12 and lower energy density on the half of the FZ closer to the device 12. The energy density in the FZ can correspond to the temperature in the FZ described below with reference to FIGS. 11A-11D. The energy levels of the first and second beams 130a, 130b can each be insufficient alone to ablate tissue (e.g., when outside an FZ). The first and second beam energy levels can combine in the FZ such that the combination of the first and second beam energy levels can be sufficient to ablate tissue. In this way, ultrasound energy can pass through a vessel wall without stenosing the vessel. The FZ can therefore be advantageously positioned outside of a blood vessel wall to prevent or otherwise minimize stenosis. For example, the first and second beams 130a, 130b can add together via constructive interference in the FZ, thereby increasing the amplitude of the ultrasound waves in the FZ as compared to the amplitude of the ultrasound waves outside of the FZ.

The transducer 16 can deliver about 6 acoustic Watts to about 14 acoustic Watts of ultrasound energy. For example, the first emitter surface 18a can deliver about 3 acoustic Watts to about 7 acoustic Watts of ultrasound energy and the second emitter surface 18b can deliver about 3 acoustic Watts to about 7 acoustic Watts of ultrasound energy. The total power delivered to the FZ can be, for example, about 6 acoustic Watts to about 14 acoustic Watts of ultrasound energy. The minimum power per area of each side of the transducer 16 can range from about 133 W/in.² (e.g., for a 3 Watt output and a transducer outer diameter 16_{OD} of about 0.170 inches) to about 4,244 W/in.² (e.g., for a 3 Watt output and a transducer outer diameter 16_{OD} of about 0.030 inches). The maximum power per area of each side of the transducer 16 can range from about 308 W/in.² (e.g., for a 7 Watt output and a transducer outer diameter 16_{OD} of about 0.170 inches) to about 9,903 W/in.² (e.g., for a 7 Watt output and a transducer outer diameter 16_{OD} of about 0.030 inches). For example, the power per area of each side of the transducer 16 can be from about 597 W/in.² to about 1,393 W/in.² (e.g., for a 3 W to 7 W output from a transducer 16 having a transducer outer diameter 16_{OD} of about 0.080 inches).

The entire FZ or a portion of the FZ can have an energy level sufficient to ablate tissue. For example, an energy dense center of the FZ can ablate tissue but a less energy dense periphery of the FZ can have a density insufficient to ablate tissue. For example, a center of the FZ can correspond to about 41.0 degrees Celsius or greater and a peripheral portion of the FZ can correspond to about 40.0 degrees Celsius or less. The temperature associated with the ultrasound energy outside the FZ can range from about 37.0 degrees Celsius to about 40.9 degrees Celsius (e.g., about 40.1 degrees Celsius). The temperature associated with the ultrasound energy inside the FZ can range from about 37.0 degrees Celsius to about 100.0 degrees Celsius, from about 41.0 degrees Celsius to about 90.0 degrees Celsius, from about 55.0 degrees Celsius to about 85.0 degrees Celsius, or combinations of these minimums and maximums.

Although FIG. 8A shows that the first focal zone FZ₁ can be moved radially and longitudinally to FZ₂, it will be appreciated that the first focal zone FZ₁ can be adjusted radially inward and/or radially outward relative to the position shown in FIG. 8A while keeping the longitudinal position of the FZ relative to the blood vessel (e.g., ablation site) and/or the device 12 the same. Likewise, it will also be appreciated that the first focal zone FZ₁ can be adjusted longitudinally toward the proximal end (including past the proximal end) and/or toward the distal end (including past the distal end) of the device 12 relative to the position shown in FIG. 8A while keeping the radial position of the FZ relative to the blood vessel (e.g., ablation site) and/or the device 12 the same.

Although not shown in FIG. 8A, one or more retractable covers can be attached to the one or more transducers (e.g., transducer 16) and/or to at least one of the reflectors (e.g., first and/or second reflectors 22a, 22b). The retractable cover(s) can partially or completely block ultrasound energy from passing through. The retractable cover(s) can be moved to partially or entirely cover the surfaces of the transducer to and reflector to make the one or more focal zones FZ larger or smaller, and/or to change the energy density profile in the one or more focal zones FZ. In lieu of or in addition to the retractable transducer and/or reflector covers, one or more retractable covers can be attached to the device such that a retractable cover can slide along the surface (or closely adjacent to the surface) of the shield 32.

FIG. 8B illustrates that the focal zones FZ (e.g., FZ₁ and FZ₂) can be asymmetrical about axes parallel and perpendicular to the longitudinal axis 40 when viewed in cross-section. The focal zone FZ₁ in FIG. 8B can be smaller than the focal zone FZ₁ in FIG. 8A, at least by virtue of the asymmetry illustrated in FIG. 8B.

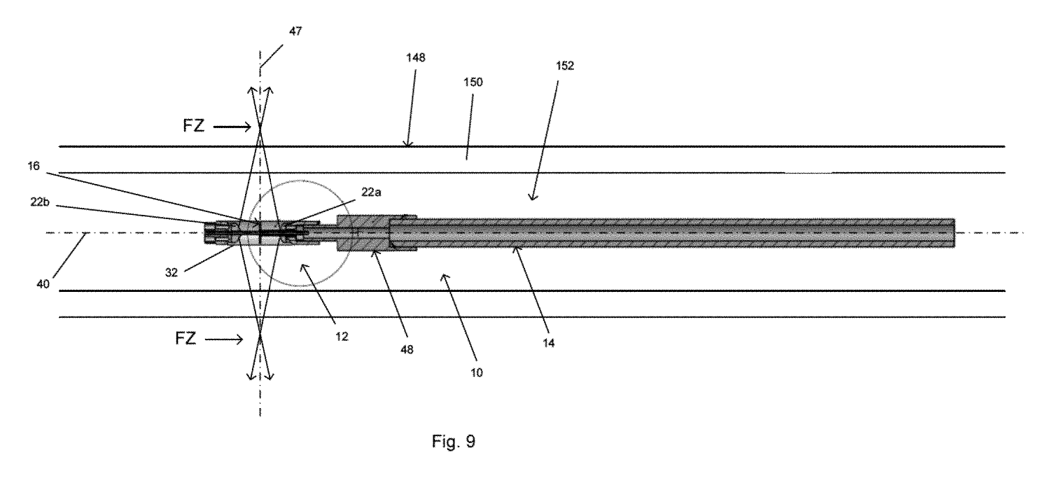

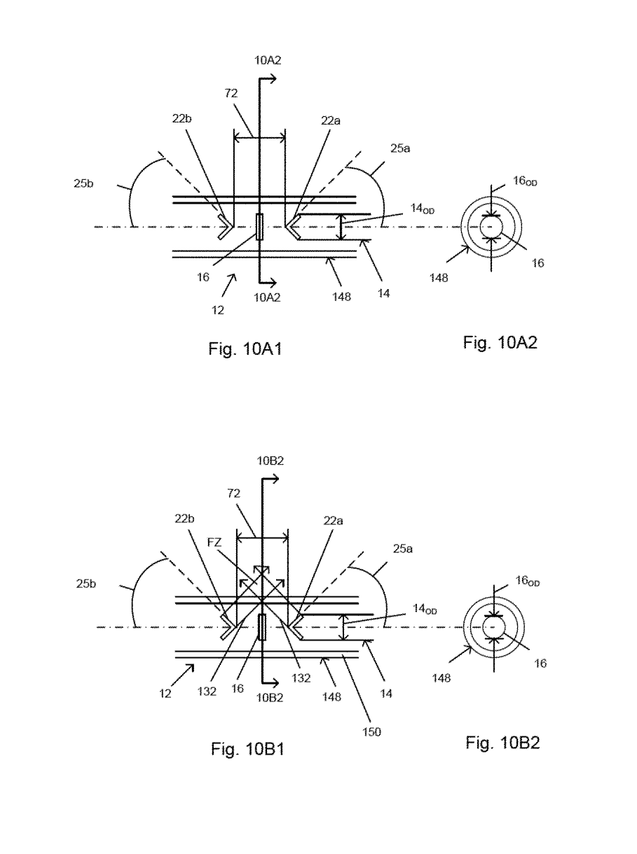

FIG. 9 illustrates that the ultrasound catheter system 10 of FIGS. 1A-1P can be used in a blood vessel 148. The catheter 14 and the ultrasound device 12 can be in a lumen 152 defined by a blood vessel wall 150. The blood vessel wall 150 can be, for example, a wall of the left or right renal artery. FIG. 9 illustrates that the FZ can extend at least partially around the longitudinal axis 40 in a plane 47. The plane 47 can be perpendicular or at an angle relative to the longitudinal axis 40. For example, FIG. 9 illustrates that the plane 47 can be perpendicular to the longitudinal axis 40. Tissue can be ablated in the FZ. Nerve tissue can be ablated in the FZ when the device 12 is emitting ultrasound energy. As shown, the FZ can be outside the vessel wall 150. This can advantageously prevent or otherwise minimize stenosis of the blood vessel. The FZ shown in FIG. 9 can be adjusted radially inward and/or outward relative to the position shown. The FZ shown in FIG. 9 can be adjusted longitudinally toward the distal end of the device 12 and/or the proximal end of the device 12.

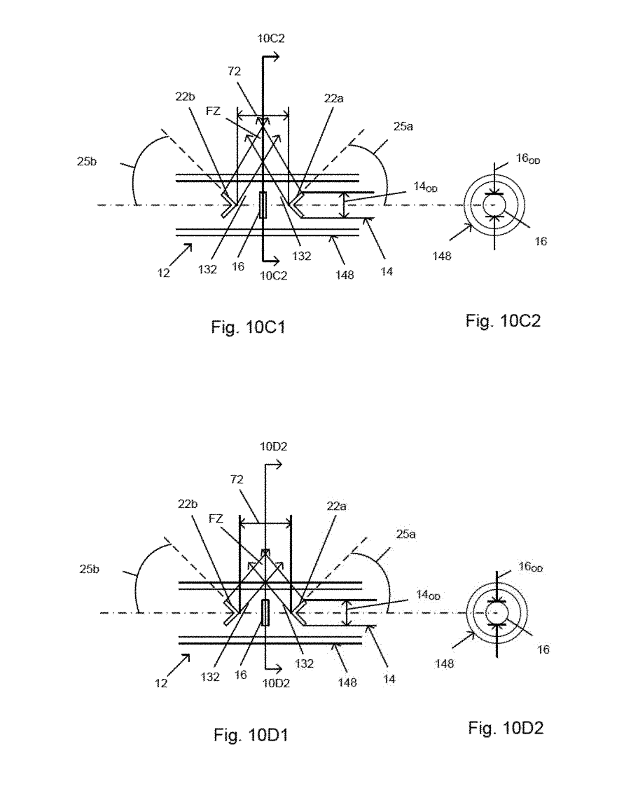

FIGS. 10A1-10D2 illustrates transverse and longitudinal cross-sectional views of a variation of an ultrasound catheter system 10 in a blood vessel 148. FIGS. 10B1-10D2 illustrate the systems 10 in a blood vessel 148. The size and/or location of the FZ relative to the device 12 can depend on, for example, the transducer frequency, the transducer diameter (e.g., transducer outer diameter 16_{OD}), the offset gap (e.g., offset gap 72, which is the distance between the first and second reflectors 22a, 22b), the reflector angle (e.g., first and second reflector angles 25a, 25b), and/or the reflector diameter (e.g., first and second reflector outer diameters 22a_{OD}, 22b_{OD}. FIGS. 10A1-10D2 each illustrates an ultrasound catheter system 10 in a blood vessel 148 having an outer diameter of about 0.240 inches and an inner diameter of about 0.180 inches. FIGS. 10A1-10D2 each illustrates that the transducer 16 can have a transducer outer diameter 16_{OD} of about 0.080 inches; however, as described above, the transducer 16 can have a transducer outer diameter 16_{OD} from, for example, about 0.030 inches to about 0.170 inches. FIGS. 10A1-10D2 each illustrates that the first and second reflectors 22a, 22b can have the same outer diameter 22a_{OD}, 22b_{OD} as the transducer 16; however, the first and second reflectors 22a, 22b can have a different outer diameter as well (e.g., larger or smaller than the transducer outer diameter). The first and second reflectors 22a, 22b can have the same or different inner and/or outer diameters relative to one another. FIGS. 10A1, 10131, 10C1, and 10D1 illustrate that the catheter 14 can have a catheter outer diameter 14_{OD} of about 0.100 inches. For illustrative purposes only, the FZ is shown on only one side of the device 12 in FIGS. 10B1, 10C1, and 10D1. As described above, the FZ can extend at least partially around the device 12 in the shape of a toroid or ring (or similar shape).

FIG. 10A1 illustrates that the first and second reflector surface angles 25a, 25b can be about 45 degrees relative to the longitudinal axis 40 and that the offset gap 72 between the first and second reflectors 22a, 22b can be about 0.210 inches.

FIG. 10B1 illustrates that the first and second reflector surface angles 25a, 25b can be about 40 degrees relative to the longitudinal axis 40 and that the offset gap 72 between the first and second reflectors 22a, 22b can be about 0.210 inches. As shown, a portion of the FZ in FIG. 10B can overlap with the vessel wall 150.

FIG. 10C1 illustrates that the first and second reflector surface angles 25a, 25b can be about 30 degrees relative to the longitudinal axis 40 and that the offset gap 72 between the first and second reflectors 22a, 22b can be about 0.210 inches. As shown, the FZ in FIG. 10C1 is completely outside of the blood vessel 148. The FZ in FIG. 10C1 is shown farther from the blood vessel 148 than in FIG. 10B1.

FIG. 10D1 illustrates that the first and second reflector surface angles 25a, 25b can be about 30 degrees relative to the longitudinal axis 40 and that the offset gap 72 between the first and second reflectors 22a, 22b can be about 0.150 inches. As shown, the FZ in FIG. 10D1 is closer to the blood vessel 148 than in FIG. 10C1.

The device(s) 12 shown in FIGS. 10A1-10D2 can be variations of a single device 12 or can be multiple different devices 12. For example, the various first and second reflector surface angles 25a, 25b and offset gaps 72 in FIGS. 10A1-10D2 can be exemplary different configurations of a single device 12.

FIGS. 11A-11D illustrate that the FZ can form a toroid outside the vessel wall 150. FIGS. 11A-11D illustrate that the FZ can have a ring shape. FIGS. 11A-11D illustrate that the FZ can have toroid shape. The FZ can be one toroid or one or more toroids combined together. For example, the energy emitted from the first transducer 16a and subsequently reflected by the first and third reflectors 22a, 22c can form a first toroid and the energy emitted from the second transducer 16b and subsequently reflected by the second and fourth reflectors 22b, 22d can form a second toroid. The first and second toroids can form the focal zone FZ. The first and second toroids can approximate the shape of a toroid. At least a portion of the first and second toroids can overlap with one another to form the FZ. The formation of multiple toroids (e.g., overlapping toroids) can advantageously extend the FZ over a greater longitudinal dimension than if only a single toroid were formed. FIGS. 11A-11D illustrate that the FZ can have a temperature gradient, with the highest temperature being located in the center of the FZ and the lowest temperature being located on the periphery of the FZ. The highest temperature can be anywhere in the FZ. The lowest temperature can be anywhere in the FZ. The temperature of the FZ can range from about 37.0 degrees Celsius to about 100.0 degrees Celsius (e.g., 82.5 degrees Celsius). For example, FIGS. 11A-11D illustrate that the highest temperature in the FZ (e.g., in the center of the FZ) can be about 82.5 degrees Celsius and that the lowest temperature in the FZ (e.g., on the periphery of the FZ) can be about 55.7 degrees Celsius. The desired therapeutic temperature can be achieved by setting the ultrasonic frequencies, power, and/or time of ablation of the one or more transducers 16. The power can be power in Watts.

The device 12 can deliver ultrasound energy to tissue in one or more focal zones over one or multiple time periods (also referred to as treatment times, burn times, ablation times, or times of ablation). The time periods can be from about 3 seconds to about 60 seconds, from about 5 seconds to about 7 seconds, and/or from about 5 seconds to about 30 seconds. For example, the device 12 can deliver ultrasound energy to tissue in the FZ for about 5 seconds. For example, the device 12 can deliver ultrasound energy to tissue in a first FZ for about 5 seconds and to tissue in a second FZ for about 7 seconds. A higher temperature can correspond to a shorter ablation time relative to a lower temperature and a lower temperature can correspond to a longer ablation time relative to a higher temperature. The temperature in the FZ can be constant (e.g., 75 degrees) or varied (e.g., 80 degrees for 2 seconds followed by degrees for 4 seconds or vice versa), for example, during an ablation period. As discussed above, the tissue in the FZ can be treated with ultrasound energy to heat tissue in the FZ to a target temperature of about 37.0 degrees Celsius to about 100.0 degrees Celsius (e.g., 82.5 degrees Celsius) to ablate the tissue. The device 12 can deliver ultrasound energy to tissue in the FZ with one or more energy levels. The device 12 can deliver continuous ultrasound waves and/or pulsed ultrasound waves to tissue in the FZ. The frequency, amplitude, phase and/or power of the ultrasound waves being emitted from the one or more transducers 16 can be adjusted. For example, the device 12 can automatically adjust the frequency, amplitude, phase and/or power of the ultrasound waves being emitted from the one or more transducers 16 based at least partly on feedback received from one or more sensors. For example, the device 12 can automatically adjust the frequency, amplitude, phase and/or power of the ultrasound waves being emitted from the one or more transducers 16 based at least partly on feedback received from one or more sensors to keep the temperature in the FZ within about 0.5 degrees Celsius to about 5.0 degrees Celsius of the target temperature (e.g., within 1.0 degrees Celsius of a target temperature of 80 degrees Celsius). Multiple focal zones can be ablated simultaneously or sequentially. Multiple focal zones can have the same or different treatment times. Multiple focal zones can have the same or different levels of ultrasound energy delivered (e.g., the same or different frequency, amplitude, phase and/or power).

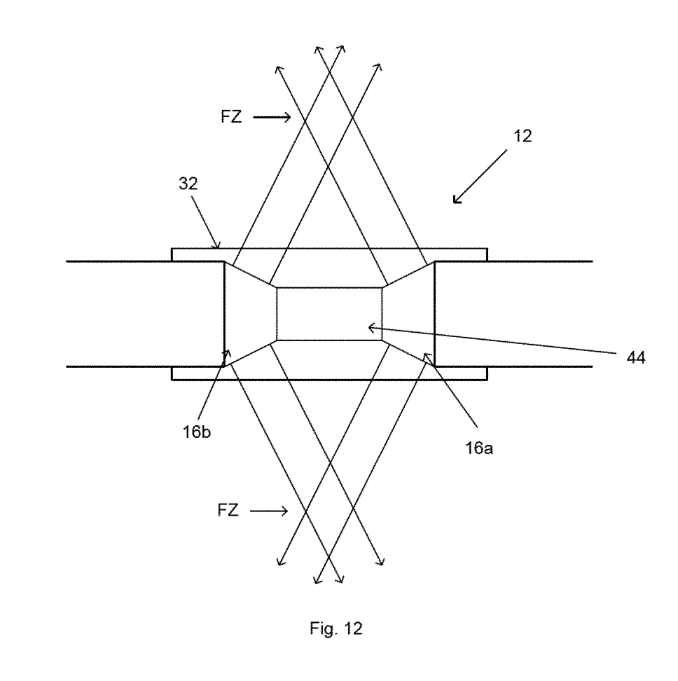

FIG. 12 illustrates that the ultrasound device 12 of FIG. 3 can focus ultrasound energy to a focal zone FZ.

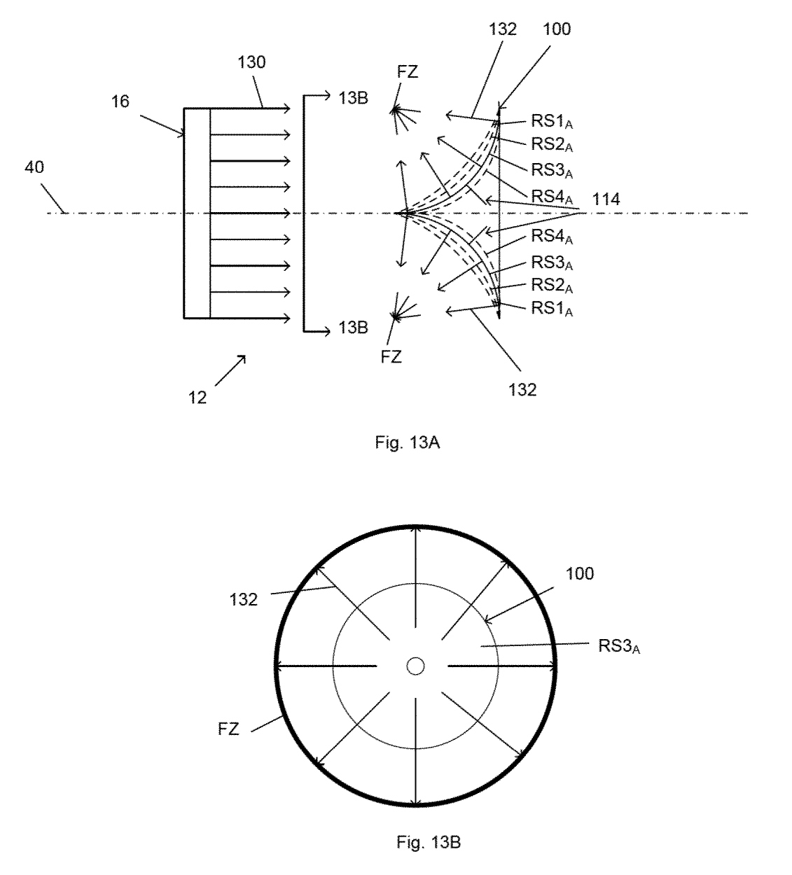

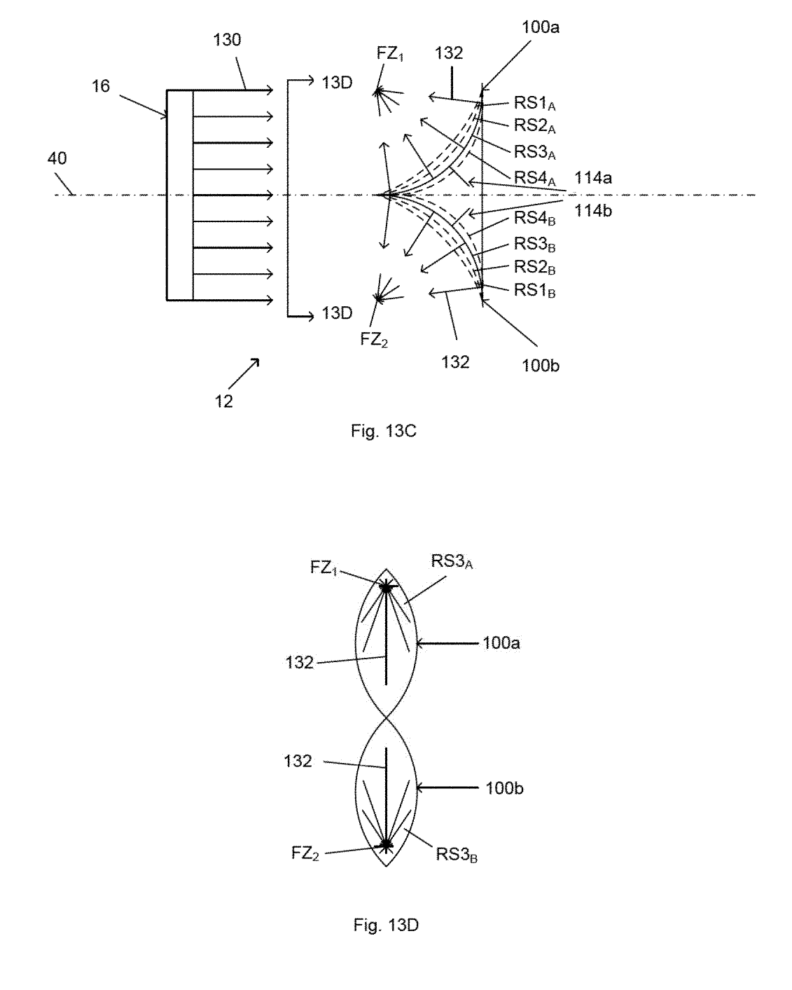

FIGS. 13A and 13B illustrate that emitted energy 130 can be redirected by the adjustable reflector 100. The redirected energy 132 can converge at a focal zone FZ. The FZ can be a ring or a toroid (or similar shape), for example, as shown in FIG. 13B. The FZ can have an adjustable focal length from the longitudinal axis 40 as described above. The focal length can be different for each of the reflective surfaces RS1_{A}-RS4_{A}, with the reflective surface RS1_{A} having the smallest focal length and the reflective surface RS4_{A} having the largest focal length.

FIGS. 13C and 13D illustrate that the device 12 can have a first adjustable reflector 100a and a second adjustable reflector 100b. FIG. 13C illustrates that emitted energy 130 can be redirected by the first and second adjustable reflectors 100a, 100b. The redirected energy 132 can converge at first and second focal zones FZ₁ and FZ₂. The first and second focal zones FZ₁ and FZ₂ can have an adjustable focal length from the longitudinal axis 40 as described above. The first focal zone FZ₁ can have an adjustable first focal length. The first focal length can be different for each of the reflective surfaces RS1_{A}-RS4_{A}, with the reflective surface RS1_{A} having the smallest first focal length and the reflective surface RS4_{A} having the largest first focal length. The second focal zone FZ₂ can have an adjustable second focal length. The second focal length can be different for each of the reflective surfaces RS1_{B}-RS4_{B}, with the reflective surface RS1_{B} having the smallest second focal length and the reflective surface RS4_{B} having the largest second focal length. The first and second focal lengths can be adjusted to be the same and/or different from one another. The first and second focal lengths can be the same and/or different from one another.

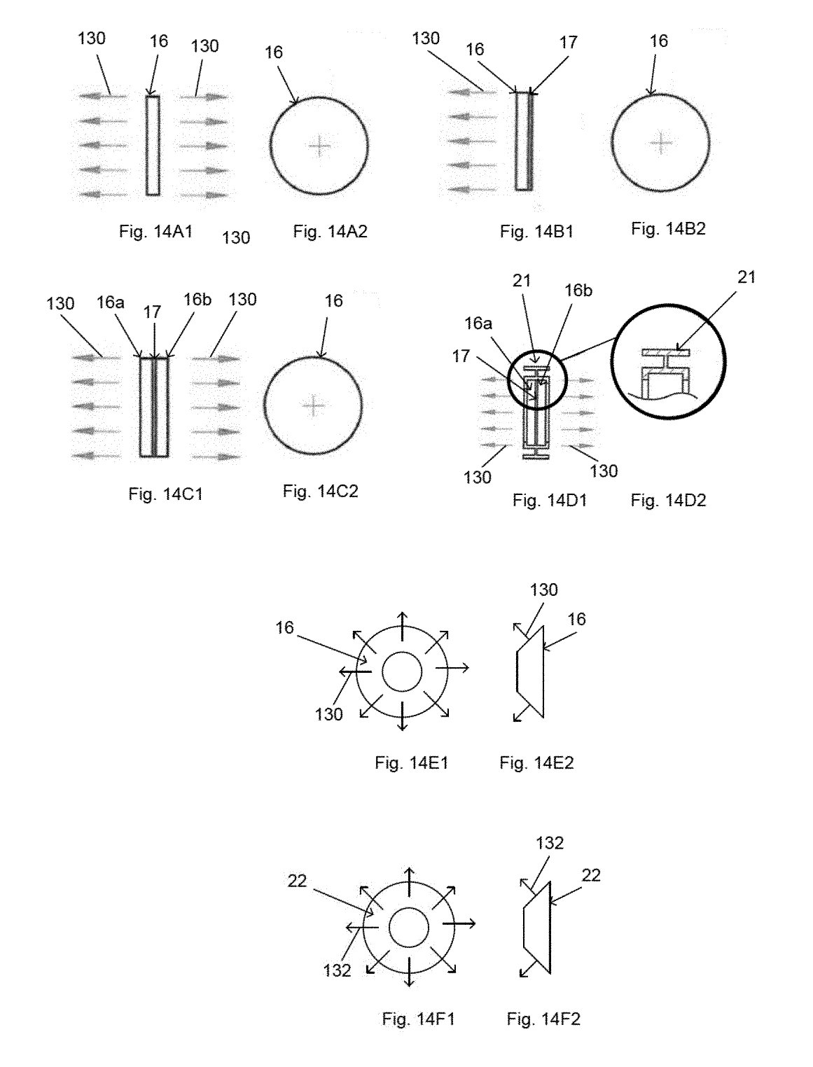

FIGS. 14A1-14E2 illustrate that the transducers 16 of FIGS. 7A1 and 7E2 can emit ultrasound energy 130. FIGS. 14F1 and 14F2 illustrate the reflectors 22 of FIGS. 7F1 and 7F2 redirecting the ultrasound energy 130. FIGS. 14F1 and 14F2 illustrate that the redirected energy 132 can converge at first and second focal zones FZ₁ and FZ₂.

The ultrasound energy can be delivered to a target ablation site with time phase frequency changes to focus the energy on the target ablation site.

The ultrasound energy can be delivered to a target ablation site continuously over a period of time or in a pulsed pattern over a period of time.

The device 12 can be moved longitudinally in a vessel to treat more than one tissue site. For example, the device 12 can be translated through the lumen of a vessel while emitting ultrasound energy, also referred to as a “drag and burn” technique.

The above disclosure focuses on the delivery of ultrasound energy; however, the device 12 can be used to deliver one or more energy sources, for example, ultrasound energy, RF energy, laser energy, microwave energy, direct heating, or combinations thereof.

The claims are not limited to the exemplary embodiments shown in the drawings, but instead may claim any feature disclosed or contemplated in the disclosure as a whole. Any elements described herein as singular can be pluralized (i.e., anything described as “one” can be more than one). Any species element of a genus element can have the characteristics or elements of any other species element of that genus. Some elements may be absent from individual figures for reasons of illustrative clarity. The above-described configurations, elements or complete assemblies and methods and their elements for carrying out the disclosure, and variations of aspects of the disclosure can be combined and modified with each other in any combination. All devices, apparatuses, systems, and methods described herein can be used for medical (e.g., diagnostic, therapeutic or rehabilitative) or non-medical purposes.

I claim:

1. An ultrasound ablation device having a longitudinal axis comprising:
a first emitter surface; and
a first conical reflector comprising a first reflective surface, wherein the first conical reflector is at a first longitudinal end of the device.

2. The device of claim 1, further comprising:
a catheter;
a second emitter surface;
a second conical reflector comprising a second reflective surface, wherein the second conical reflector is at a second longitudinal end of the device;
a cylindrical shield, wherein at least a portion of the cylindrical shield is transparent such that less than 10% of ultrasound energy that passes therethrough is attenuated; and
an ultrasound coupling chamber, wherein the first and second emitter surfaces, the first and second reflective surfaces, and a coupling fluid are in the ultrasound coupling chamber, wherein the coupling fluid comprises saline, wherein the ultrasound coupling chamber has a coupling fluid inlet and a coupling fluid outlet in fluid communication with the ultrasound coupling chamber, wherein the ultrasound coupling chamber is at least partially defined by the cylindrical shield,
wherein the first and second emitter surfaces are flat,
wherein an axis normal to the first emitter surface extends at least partially in a first longitudinal direction toward the first longitudinal end of the device and wherein an axis normal to the second emitter surface extends at least partially in a second longitudinal direction toward the second longitudinal end of the device,
wherein the first and second emitter surfaces are surfaces of a piezoelectric transducer configured to have a harmonic frequency from 9 MHz to 15 MHz,
wherein the first reflective surface extends around the longitudinal axis and wherein the second reflective surface extends around the longitudinal axis,
wherein the first reflective surface is at a first reflector surface angle relative to the longitudinal axis, wherein the first reflector surface angle is from 30 degrees to 60 degrees, wherein the second reflective surface is at a second reflector surface angle relative to the longitudinal axis, and wherein the second reflector surface angle is from 30 degrees to 60 degrees, and
wherein the device has a first configuration and a second configuration, wherein at least one of the first and second conical reflectors is closer to at least one of the first and second emitter surfaces in the first configuration than in the second configuration.

3. The device of claim 2, further comprising a coupling fluid source in fluid communication with the coupling fluid inlet, wherein the coupling fluid first outlet is in fluid communication with an outside environs.

4. The device of claim 3, wherein the coupling fluid outlet comprises a recirculation port in fluid communication with the coupling fluid source and/or another coupling fluid source.

5. The device of claim 2, wherein the first and second conical reflectors comprise first and second frusto-conical reflectors.

6. The device of claim 2, wherein the first conical reflector is moveable relative to the second conical reflector, or wherein the second conical reflector is moveable relative to the first conical reflector, or wherein at least one of the first and second conical reflectors is movable relative to at least one of the first and second emitter surfaces, or wherein at least one of the first and second emitter surfaces is movable relative to at least one of the first and second conical reflectors, or wherein the first and second conical reflectors and the first and second emitter surfaces are each movable relative to one another.

7. An ultrasound ablation device having a longitudinal axis comprising:
a transducer comprising a first emitter surface; and
a first conical reflector comprising a first reflective surface, wherein the first conical reflector is at a first longitudinal end of the device.

8. The device of claim 7, further comprising:
a second emitter surface; and
a second conical reflector comprising a second reflective surface, wherein the second conical reflector is at a second longitudinal end of the device,
wherein the device has a first configuration and a second configuration, wherein the first and second conical reflectors are longitudinally closer in the first configuration than in the second configuration.

9. The device of claim 8, wherein the first reflective surface extends at least partially around the longitudinal axis and wherein the second reflective surface extends at least partially around the longitudinal axis.

10. The device of claim 8, wherein the first conical reflector is moveable relative to the second conical reflector, or wherein the second conical reflector is moveable relative to the first conical reflector, or wherein at least a portion of at least one of the first and second conical reflectors is movable relative to at least one of the first and second emitter surfaces, or wherein at least one of the first and second emitter surfaces is movable relative to at least one of the first and second conical reflectors, or wherein the first and second conical reflectors and the first and second emitter surfaces are each movable relative to one another.

11. A method of performing ultrasound ablation with an ablation device having a longitudinal axis, the method comprising:
emitting, from a first emitter surface, a first ultrasound energy beam in a first beam first direction at a first beam first angle relative to the longitudinal axis; and
redirecting, via a first conical reflector, the first ultrasound energy beam.

12. The method of claim 11, further comprising:
redirecting, via a first conical reflector, the first ultrasound energy beam from the first beam first direction to a first beam second direction, wherein the first beam second direction extends at least partially in a radial direction away from the longitudinal axis at a first beam second angle relative to the longitudinal axis, and wherein the first conical reflector comprises a first reflector surface;
emitting, from a second emitter surface, a second ultrasound energy beam in a second beam first direction at a second beam first angle relative to the longitudinal axis; and
redirecting, via a second conical reflector, the second ultrasound energy beam from the second beam first direction to a second beam second direction, wherein the second beam second direction extends at least partially in a radial direction away from the longitudinal axis at a second beam second angle relative to the longitudinal axis, and wherein the second conical reflector comprises a second reflector surface.

13. The method of claim 12, wherein the first beam extends at least partially around the longitudinal axis in the first beam first direction and in the first beam second direction, and wherein the second beam extends at least partially around the longitudinal axis in the second beam first direction and in the second beam second direction.

14. The method of claim 12, wherein the first beam extends toward a focal zone in the first beam second direction, wherein the second beam extends toward the focal zone in the second beam second direction, and wherein at least a portion of the first and second beams converge in the focal zone.

15. The method of claim 14, wherein the first beam comprises a first beam energy level, wherein the second beam comprises a second beam energy level wherein the focal zone has focal zone energy levels greater than each of the first and second beam energy levels, and wherein the focal zone energy levels are sufficient to ablate tissue.

16. The method of claim 14, further comprising moving the focal zone from a first focal zone to a second focal zone.

17. The method of claim 16, wherein moving the focal zone from the first focal zone to the second focal zone comprises at least one of:
moving the first reflector surface relative to at least one of the first emitter surface, the second emitter surface, and the second reflector surface;
moving the second reflector surface relative to at least one of the first emitter surface, the second emitter surface, and the first reflector surface;
moving the first emitter surface relative to at least one of the second emitter surface, the first reflector surface, and the second reflector surface; and
moving the second emitter surface relative to at least one of the first emitter surface, the first reflector surface, and the second reflector surface.

18. The method of claim 14, wherein the focal zone has an adjustable focal length from the longitudinal axis.

19. The method of claim 18, wherein the focal length is adjustable from 2 mm to 6 mm from the longitudinal axis.

20. The method of claim 14, wherein the focal zone has a toroid shape or a ring shape that extends at least partially around the longitudinal axis.
